(19) United States
(12) Patent Application Publication
(10) Pub. No.: US 2015/0201649 A1
(43) Pub. Date: Jul. 23, 2015

(54) ALGAL-BASED ANIMAL FEED COMPOSITION, ANIMAL FEED SUPPLEMENT, AND USES THEREOF

(71) Applicant: CORNELL UNIVERSITY, Ithaca, NY (US)

(72) Inventor: Xingen Lei, Ithaca, NY (US)

(73) Assignee: CORNELL UNIVERSITY, Ithaca, NY (US)

(21) Appl. No.: 14/415,013

(22) PCT Filed: Jul. 17, 2013
(86) PCT No.: PCT/US13/50827

§ 371 Date: Jan. 15, 2015

Related U.S. Application Data
(60) Provisional application No. 61/672,581, filed on Jul. 17, 2012.
(60) Provisional application No. 61/714,509, filed on Oct. 16, 2012.
(60) Provisional application No. 61/823,722, filed on May 15, 2013.

(51) Int. Cl. A23K 1/16; A23K 1/14; A23K 1/17; A23K 1/175; A23K 1/18; A61K 36/899; A61K 36/48; A61K 36/02; A61K 33/42; A61K 33/14; A61K 31/198; A61K 35/12; A61K 38/02; A61K 33/06; A61K 45/06; A61K 33/34; A61K 33/04; A61K 33/30; A61K 33/18; A61K 33/32; A61K 33/26; A23K 1/06

(52) U.S. Cl. CPC A23K 1/1631; A23K 1/06; A23K 1/143; A23K 1/17; A23K 1/1634; A23K 1/1751; A23K 1/175; A23K 1/1603; A23K 1/1753; A23K 1/1758; A23K 1/164; A23K 1/184; A61K 36/899; A61K 36/48; A61K 36/02; A61K 33/42; A61K 33/14; A61K 31/198; A61K 35/12; A61K 38/02; A61K 33/06; A61K 45/06; A61K 33/34; A61K 33/04; A61K 33/30; A61K 33/18; A61K 33/32; A61K 33/26; A61K 35/16

(57) ABSTRACT

The present invention relates to an animal feed composition comprising one or more grains in an amount totaling 50-70% w/w of the composition; a non-algal protein source in an amount totaling 15-30% w/w of the composition; algae in an amount totaling 3-15% w/w of the composition; an oil heterologous to the algae in an amount totaling 0.5-15% w/w of the composition; an inorganic phosphate source in an amount totaling up to 1.5% w/w of the composition; a sodium source in an amount totaling up to 0.5% w/w of the composition; and one or more amino acids selected from the group consisting of lysine, threonine, isoleucine, tryptophan, and methionine in an amount totaling up to 0.5% w/w of the composition. Also disclosed are an animal feed supplement, methods of feeding animals, methods of improving the feed efficiency of an animal, and an improvement to animal feed.

This application claims the benefit of U.S. Provisional Patent Application Ser. No. 61/672,581, filed Jul. 17, 2012; U.S. Provisional Patent Application Ser. No. 61/714,509, filed Oct. 16, 2012; and U.S. Provisional Patent Application Ser. No. 61/823,722, filed May 15, 2013, which are hereby incorporated by reference in their entirety.

### FIELD OF THE INVENTION

The present invention relates to algal-based animal feed compositions, animal feed supplements, and uses thereof.

### BACKGROUND OF THE INVENTION

Although fossil fuels are the major source of energy for heating, transportation, manufacturing, and the generation of electricity, these fuels are non-renewable. Therefore, the search for renewable energy sources has become a key challenge of this century. Many species of microalgae contain large amounts of lipids that are suitable for the production of biofuels, especially biodiesel (Gouveia et al, “Microalgae as Raw Material for Biofuels Production,” J. Ind. Microbiol. Biotechnol. 36:269-274 (2009)). Microalgae are the natural food source for many important aquaculture species such as molluscs, shrimps, and fish (Spolaore et al., “Commercial Applications of Microalgae,” J. Biosci. Bioeng. 101(2):87-96 (2006)), and several species of microalgae have been reported to be acceptable for inclusion in diets for swine, rabbits, broiler chickens, laying hens, and ruminant animals (Becker W. In: “Handbook of Microalgal Culture: Biotechnology and Applied Phycology,” Richmond, A. (Ed). Microalgae in Human and Animal Nutrition, Oxford, Blackwell Science, pp. 312-351 (2004); Becker, E. W., “Micro-Algae as a Source of Protein,” Biotech. Adv. 25:207-210 (2007)).

Soybean meal is a by-product of the extraction of oil from soybeans. Having high protein content, it serves as the main protein source for poultry and other food-producing animals. Due to increasing human demand for soybean products, soybean meal is becoming more expensive and limited in supply (U.S. Department of Agriculture, Economic Research Service. Economics, Statistics, and Market Information System. Oil Crops, Situation and Outlook Yearbook, Washington, D.C. (2011); Mielke, T., “Major Challenges Ahead: World Soybean Supply and Demand Outlook for 2012/13,” Int. News on Fats, Oils and Related Materials (INFORM) 23(7):468-470 (2012)). Therefore, it is important to explore other medium-to-high protein feedstuffs for the sustainable development of animal production.

Microalgae are a rich source of protein, essential fatty acids, vitamins, and minerals (Becker W. In: “Handbook of Microalgal Culture: Biotechnology and Applied Phycology,” Richmond, A. (Ed), Microalgae in Human and Animal Nutrition, Oxford, Blackwell Science, pp. 312-351 (2004)). After lipid removal, the residual biomass contains even higher concentrations of protein and other nutrients. Microalgae are good sources of long chain polyunsaturated fatty acids (“PUFA”) and have been used to enrich diets with omega-3 PUFA (Herber et al., “Dietary Marine Algae Promotes Efficient Deposition of n-3 Fatty Acids for the Production of Enriched Shell Eggs,” Poult. Sci. 75:1501-1507 (1996); Barclay et al. In: The return of ω3 Fatty Acids into the Food Supply. I. Land-based Animal Food Products and their Health Effects, Simopoulos, A. P. (Ed). “Production of Docosahexaenoic Acid from Microalgae and Its Benefits for Use in Animal Feeds,” World Rev. Nutr. Diet. Basil, Karger 83:61-76 (1998); Nitsan et al., “Enrichment of Poultry Products with ω3 Fatty Acids by Dietary Supplementation with the Alga Nannochloropsis and Mantur Oil,” J. Agric. Food Chem. 47:5127-5132 (1999)).

The defatted algae by-product of biofuel production contains low contents of residual lipids containing long chain PUFA that may have significant nutritional value. Diatoms are a class of microalgae that characteristically accumulate amorphous silicon in their membranes resulting in distinct external features called frustules (Martin-Jézéquel et al., “Silicon Metabolism in Diatoms: Implications for Growth,” J. Phycol. 36:821-840 (2000)). They have not been used in nutritional studies with animals but have been under investigation as a source of lipids for biofuel production. The defatted diatom biomass contains protein, residual fat, carbohydrates, silicon, and a large mineral fraction that includes calcium, phosphorus, sodium, potassium, chloride, and several other nutritionally significant minerals (Becker W. In: “Handbook of Microalgal Culture: Biotechnology and Applied Phycology,” Richmond, A. (Ed), Microalgae in Human and Animal Nutrition, Oxford, Blackwell Science, pp. 312-351 (2004); Harrison et al., “Response of the Marine Diatom, Thalassiosira weissflogii, to Iron Stress,” Limnol. Oceanogr. 3(5):989-997 (1986); Taraldsvik et al., “The Effect of pH on Growth Rate, Biochemical Composition and Extracellular Carbohydrate Production of the Marine Diatom, Skeletonema costatum,” Eur. J. Phycol. 35:189-194 (2000); Pahl et al., “Growth Dynamics and the Proximate Biochemical Composition and Fatty Acid Profile of the Heterotrophically Grown Diatom Cyclotella cryptica.,” J. Appl. Phycol. 22:165-171 (2010)).

The global population is expected to reach 9 billion by the year 2050. Thus, the plant breeding community has been working towards doubling crop yields to keep up with future food demands (World Population Prospects, 2007, “World Population Prospects, the 2006 Revision, Highlights, Working Paper No. ESA/P/WP.202.,” United Nations, Department of Economic and Social Affairs, Population Division, New York). This might be very challenging as agricultural land is shrinking, global water tables are depleting (Brown, L., Outgrowing the Earth, a Food Security Challenge in Age of Falling Water Tables and Rising Temperatures, New York. W. W. Norton (2004)) and crop inputs, especially chemical fertilizers, are reducing as a means of minimizing greenhouse gas emissions and the agricultural carbon footprint (Food and Agricultural Organization, In World Agriculture: Towards 2015/2030. Summary Report. Prospects for the Environment: Agriculture and the Environment, Rome (2002)). Meanwhile, food-producing animals rely heavily on soybean meal and corn to meet their protein and energy requirements, creating a direct competition of these two foods for human consumption. This competition will only exacerbate the future food demand as meat consumption in developing countries increases. Therefore, alternative protein and energy sources are required to replace soybean meal and corn in animal feeds for sustainable animal agriculture.

Broiler chicks are the fastest growing and most efficient food species that is consumed worldwide. While the domestic broiler industry produces 36 billion pounds of meat with $22 billon value (USDA-National Agriculture Statistics Service, Poultry-Production and Value, 2012 Summary (April 2013), ISSN: 1949-1573 (2013)), it also uses up 13.5 and 30 million metric tons of soybean meal and corn per annum, respectively. Various algae have been tested as sources of protein for broiler chicks by replacing soybean meal or fish meal (Grau et al., “Sewage-Grown Algae as a Feedstuff for Chicks,” Poult. Sci. 36:1046-1051 (1957); Lipstein et al., “The Nutritional Value of Sewage-Grown, Alum Flocculated Micractinium Algae in Broiler and Layer Diets,” Poult. Sci. 60:2628-2638 (1981); Combs, G. F., “Algae (Chlorella) as a Source of Nutrients for the Chick,” Science 116:453-454 (1952); Mokady et al., “Algae Grown on Wastewater as a Source of Protein for Young Chickens and Rats,” Nutr. Rep. Int. 19:383-390 (1979); Lipstein et al., “The Nutritional Value of Sewage-Grown Samples of Chlorella and Micractinium in Broiler Diets,” Poult. Sci. 62:1254-1260 (1983); Vankatarman et al., “Replacement Value of Blue-Green Alga (Spirulina platensis) for Fishmeal and a Vitamin-Mineral Premix for Broiler Chick,” Br. Poult. Sci. 35:373-38 (1994)). Dietary levels from 5% to 10% algae substituted safely in partial replacement of these conventional ingredients. (Lipstein et al., “The Nutritional Value of Sewage-Grown, Alum Flocculated Micractinium Algae in Broiler and Layer Diets,” Poult. Sci. 60:2628-2638 (1981); Ross et al., “The Nutritional Value of Dehydrated, Blue-Green Algae (Spirulina plantensis) for Poultry,” Poult. Sci. 69:794-800 (1990); Lipstein et al., “The Nutritional Value of Sewage-Grown Samples of Chlorella and Micractinium in Broiler Diets,” Poult. Sci. 62:1254-1260 (1983)). Similar results were seen in swine (Hintz et al., “Sewage-Grown Algae as a Protein Supplement for Swine,” Animal Production 9:135-140 (1967)). However, higher levels of inclusion (20%) led to adverse effects on performance in poultry, (Mokady et al., “Algae Grown on Wastewater as a Source of Protein for Young Chickens and Rats,” Nutr. Rep. Int. 19:383-390 (1979)) probably due to relative deficiency in the sulfur-containing amino acids methionine and cysteine (Becker, W., In: Handbook of Microalgal Culture: Biotechnology and Applied Phycology. Richmond, A. (Ed). Microalgae in Human and Animal Nutrition, Oxford, Blackwell Science, pgs. 312-351 (2004)) and/or low digestibility of microalgal protein.

Chemically, defatted microalgae are uniquely different from other microalgae. They are supposed to contain high levels of ash and silicon (Si) in their cell membranes, and have unique morphological structures known as frustules (Martin-Jézéquel et al., “Silicon Metabolism in Diatoms: Implications for Growth,” J. Phycol. 36:821-840 (2000)). Like most algae, they exhibit considerably higher sodium contents than land-based plants (Rupérez, P., “Mineral Content of Edible Marine Seaweeds,” Food Chem. 79:23-26 (2002)). Because high levels of ash (Keegan et al., “The Effects of Poultry Meal Source and Ash Level on Nursery Pig Performance,” J. Anim. Sci. 82:2750-2756 (2004)) and sodium (Gal-Garber et al., “Nutrient Transport in the Small Intestine: Na⁺, K⁺-ATPase Expression and Activity in the Small Intestine of the Chicken as Influenced by Dietary Sodium,” Poul. Sci. 82:1127-113 (2003)) and the balance of monovalent minerals (Leach et al., “Further Studies on Tibial Dyschondroplasia (Cartilage Abnormality) in Young Chicks,” J. Nutr. 102:1673-1680 (1972); Sauveur et al., “Interrelationship between Dietary Concentrations of Sodium, Potassium and Chloride in Laying Hens,” Br. Poult. Sci. 19:475-485 (1978)) affect body metabolism and health status, it remains to be determined if the defatted algae inclusion into animal diets causes toxicity or side effects such as feed refusal.

The present invention is directed to overcoming these and other deficiencies in the art.

### SUMMARY OF THE INVENTION

One aspect of the present invention relates to an animal feed composition comprising one or more grains in an amount totaling 50-70% w/w of the composition; a non-algal protein source in an amount totaling 15-30% w/w of the composition; algae in an amount totaling 3-15% w/w of the composition; an oil heterologous to the algae in an amount totaling 0.5-15% w/w of the composition; an inorganic phosphate source in an amount totaling up to 1.5% w/w of the composition; a sodium source in an amount totaling up to 0.5% w/w of the composition; and one or more amino acids selected from the group consisting of lysine, threonine, isoleucine, tryptophan, and methionine in an amount totaling up to 0.5% w/w of the composition.

Another aspect of the present invention relates to an animal feed supplement comprising algae; an inorganic phosphate source in an amount (w/w) of algae (1-25):inorganic phosphate (1-2); a sodium source in an amount (w/w) of algae (1-25):sodium (0.1-0.6); and one or more amino acids selected from the group consisting of lysine, threonine, isoleucine, tryptophan, and methionine in an amount (w/w) of algae (1-25):amino acids (3-5).

A further aspect of the present invention relates to a method of feeding an animal. This method involves administering to an animal the animal feed composition of the present invention.

Yet another aspect of the present invention relates to a method of feeding an animal. This method involves administering to an animal an animal feed in combination with the animal feed supplement of the present invention.

Yet a further aspect of the present invention relates to a method of improving the feed efficiency of an animal. This method involves administering to an animal an animal feed in combination with the animal feed supplement of the present invention under conditions effective to cause a 3-15% decrease in plasma uric acid levels in the animal relative to such animal receiving the animal feed without the animal feed supplement, thereby improving the feed efficiency in the animal.

Still another aspect of the present invention relates to a method of improving the feed efficiency of an animal. This method involves administering to an animal the animal feed composition of the present invention under conditions effective to cause a 3-15% decrease in plasma uric acid levels in the animal relative to such animal receiving an animal feed other than the animal feed composition, thereby improving the feed efficiency in the animal.

Another aspect of the present invention relates to an animal feed, where the improvement comprises algae in an amount effective to decrease uric acid levels in plasma in an animal by 3-15% after consuming the animal feed, thereby improving feed efficiency in the animal.

### BRIEF DESCRIPTION OF THE DRAWINGS

FIG. 1 is a table showing the effect of dietary defatted diatom microalgal biomass on fecal dry matter, as well as fecal and plasma minerals concentrations of pigs in Experiment 1 of Example 1 below.

FIGS. 2A-C are tables showing the effect of dietary defatted diatom microalgal biomass on fecal and plasma minerals concentrations of pigs in Experiment 1 of Example 1 below.

FIG. 3 is a table showing the effect of dietary full-fat diatom microalgal biomass on overall growth performance of pigs in Experiment 2 of Example 1 below.

FIG. 4 is a table showing the effect of dietary full-fat diatom microalgal biomass on plasma biochemical measures of pigs in Experiment 2 of Example 1 below.

FIGS. 5A-B are tables showing dietary levels of defatted diatom and selected nutrients in Experiments 1, 2, and 3 of Example 3 below.

FIG. 6 is a table showing the effects of dietary inclusion of 7.5 or 10% of defatted microalgae on growth performance of broiler chicks in Experiment 1 of Example 3 below.

FIG. 7 is a table showing the effects of dietary inclusion of 7.5 or 10% defatted microalgae on biomarkers in plasma and liver of broiler chicks at 6 weeks of age in Experiment 1 of Example 3 below.

FIG. 8 is a table showing growth performance responses of broiler chicks to the 7.5% defatted microalgae diets with manipulations of various nutrients in Experiment 2 of Example 3 below.

FIG. 9 is a table showing responses of growth performance of male broiler chicks to 7.5% defatted microalgae diets containing amino acids and protease in Experiment 3 of Example 3 below.

FIG. 10 is a table showing responses of plasma biomarkers of male broiler chicks to 7.5% defatted microalgae diets containing amino acids and protease at 3 and 6 weeks of age in Experiment 3 of Example 3 below.

### DETAILED DESCRIPTION OF THE INVENTION

The present invention relates generally to animal feed compositions and animal feed supplements containing microalgae. While the inclusion of algae in animal feed is known, the present invention relates to improved algal-based animal feed compositions and animal feed supplements that provide a combination of ingredients that improve existing animal feed.

One aspect of the present invention relates to an animal feed composition comprising one or more grains in an amount totaling 50-70% w/w of the composition; a non-algal protein source in an amount totaling 15-30% w/w of the composition; algae in an amount totaling 3-15% w/w of the composition; an oil heterologous to the algae in an amount totaling 0.5-15% w/w of the composition; an inorganic phosphate source in an amount totaling up to 1.5% w/w of the composition; a sodium source in an amount totaling up to 0.5% w/w of the composition; and one or more amino acids selected from the group consisting of lysine, threonine, isoleucine, tryptophan, and methionine in an amount totaling up to 0.5% w/w of the composition.

In one embodiment, the animal feed composition comprises one or more grains in an amount of 51-69%, 52-68%, 53-67%, 54-66%, 55-65%, 56-64%, 57-63%, 58-62%, 59-61%, or about 60% w/w of the composition. Alternatively, the animal feed composition comprises one or more grains in an amount of about 50%, 51%, 52%, 53%, 54%, 55%, 56%, 57%, 58%, 59%, 60%, 61%, 62%, 63%, 64%, 65%, 66%, 67%, 68%, 69%, or 70% w/w of the composition.

In one embodiment, the animal feed composition comprises a non-algal protein source in an amount totaling 16-29%, 17-28%, 18-27%, 19-26%, 20-25%, 21-24%, or 22-23% w/w of the composition. Alternatively, the animal feed composition comprises one or more grains in an amount of about 16%, 17%, 18%, 19%, 20%, 21%, 22%, 23%, 24%, 25%, 26%, 27%, 28%, 29%, or 30% w/w of the composition.

In one embodiment, the animal feed composition comprises algae in an amount totaling 4-14%, 5-13%, 6-12%, 7-11%, 8-10%, or about 9% w/w of the composition. Alternatively, the animal feed composition comprises algae in an amount of about 3%, 4%, 5%, 6%, 7%, 7.5%, 8%, 9%, 10%, 11%, 12%, 13%, 14%, or 15% w/w of the composition.

In one embodiment, the animal feed composition comprises an oil heterologous to the algae in an amount totaling 0.6-14%, 0.7-13%, 0.8-12%, 0.9-11%, 1-10%, 2-9%, 3-8%, 4-7%, or 5-6% w/w of the composition. Alternatively, the animal feed composition comprises oil heterologous to the algae in an amount of about 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, or 15% w/w of the composition.

In one embodiment, the animal feed composition comprises an inorganic phosphate source in an amount totaling up to 1.4%, 1.3%, 1.2%, 1.1%, 1.0%, 0.9%, 0.8%, 0.7%, 0.6%, 0.5%, 0.4%, 0.3%, 0.2%, 0.1%, 0.05%, or 0.01% w/w of the composition.

In one embodiment, the animal feed composition comprises a sodium source in an amount totaling up to 0.4%, 0.3%, 0.2%, 0.1%, 0.09%, 0.08%, 0.07%, 0.06%, 0.05%, or 0.01% w/w of the composition.

In one embodiment, the animal feed composition comprises one or more amino acids selected from the group consisting of lysine, threonine, isoleucine, tryptophan, and methionine in an amount totaling up to 0.4%, 0.3%, 0.2%, 0.1%, 0.09%, 0.08%, 0.07%, 0.06%, 0.05%, or 0.01% w/w of the composition.

Another aspect of the present invention relates to an animal feed supplement comprising algae; an inorganic phosphate source in an amount (w/w) of algae (1-25):inorganic phosphate (1-2); a sodium source in an amount (w/w) of algae (1-25):sodium (0.1-0.6); and one or more amino acids selected from the group consisting of lysine, threonine, isoleucine, tryptophan, and methionine in an amount (w/w) of algae (1-25):amino acids (3-5).

As used herein, “w/w” refers to proportions by weight, and means the ratio of the weight of one substance in a composition to the total weight of the composition, or the weight of one substance in the composition to the weight of another substance of the composition. For example, reference to a composition that comprises 15% w/w algae means that 15% of the composition's weight is composed of algae (e.g., such a composition having a weight of 100 mg would contain 5 mg of algae) and the remainder of the weight of the composition (e.g., 95 mg in the example) is composed of other ingredients. Reference to a composition that comprises an amount (w/w) of inorganic phosphate in terms of algae (1-25):inorganic phosphate (1-2) means the amount of inorganic phosphate included in the composition is relative to the amount of algae in the composition.

As used herein, the terms “microalgae” and “algae” are used interchangeably and mean a eukaryotic microbial organism that contains a chloroplast, and which may or may not be capable of performing photosynthesis. Microalgae include obligate photoautotrophs, which cannot metabolize a fixed carbon source as energy, as well as heterotrophs, which can live solely off of a fixed carbon source, including obligate heterotrophs, which cannot perform photosynthesis. Microalgae include unicellular organisms that separate from sister cells shortly after cell division, such as Chlamydomonas, as well as microbes such as, for example, Volvox, which is a simple multicellular photosynthetic microbe of two distinct cell types.

In one embodiment, the algae or microalgae is a diatom, e.g., diatom microalgae Staurosira sp. or Nannofrustulum. Diatoms are the major phytoplankton characterized by silica in the outer membrane of their cell walls. Diatoms construct ornamented shells of amorphous silica that contain complex material in their frustule structure. Studies on diatoms show that in some species, total amino acids found in the cell wall are 1.2-fold greater than those found in the cell contents. Also, certain amino acids appear to be consistently enriched in the cell wall compared to the cell contents, such as serine, threonine, and glycine.

Other microalgae may include cells such as Desmodesmus sp., Chlorella, Parachlorella, and Dunaliella. Chlorella is a genus of single-celled green algae, belonging to the phylum Chlorophyta. Chlorella cells are generally spherical in shape, about 2 to 10 μm in diameter, and lack flagella. Some species of Chlorella are naturally heterotrophic. Non-limiting examples of Chlorella species suitable for use in the animal feed and animal feed supplements of the present invention include Chlorella protothecoides, Chlorella ellipsoidea, Chlorella minutissima, Chlorella zofinienesi, Chlorella luteoviridis, Chlorella kessleri, Chlorella sorokiniana, Chlorella fusca var. vacuolata Chlorella sp., Chlorella cf. minutissima, and Chlorella emersonii. Chlorella protothecoides, is known to have a high composition of lipids.

Other species of Chlorella suitable for use in the animal feed and animal feed supplement compositions of the present invention include, without limitation, the species anitrata, Antarctica, aureoviridis, candida, capsulate, desiccate, ellipsoidea (including strain CCAP 211/42), glucotropha, infusionum (including var. actophila and var. auxenophila), kessleri (including any of UTEX strains 397, 2229, 398), lobophora (including strain SAG 37.88), luteoviridis (including strain SAG 2203 and var. aureoviridis and lutescens), miniata, mutabilis, nocturna, ovalis, parva, photophila, pringsheimii, protothecoides (including any of UTEX strains 1806, 411, 264, 256, 255, 250, 249, 31, 29, 25 or CCAP 211/8D, or CCAP 211/17 and var. acidicola), regularis (including var. minima, and umbricata), reisiglii (including strain CCP 11/8), saccharophila (including strain CCAP 211/31, CCAP 211/32 and var. ellipsoidea), salina, simplex, sorokiniana (including strain SAG 211.40B), sphaerica, stigmatophora, trebouxioides, vanniellii, vulgaris (including strains CCAP 211/11K, CCAP 211/80 and f. tertia and var. autotrophica, viridis, vulgaris, tertia, viridis), xanthella, and zofingiensis.

Other genera of microalgae can also be used in the animal feed compositions of the present invention and may include, for example, Parachlorella kessleri, Parachlorella beijerinckii, Neochloris oleabundans, Bracteacoccus, including B. grandis, B. cinnabarinas, and B. aerius, Bracteococcus sp. and Scenedesmus rebescens. Other nonlimiting examples of microalgae species include Achnanthes orientalis; Agmenellum; Amphiprora hyaline; Amphora, including A. coffeiformis including A.c. linea, A.c. punctata, A.c. taylori, A.c. tenuis, A.c. delicatissima, A.c. delicatissima capitata; Anabaena; Ankistrodesmus, including A. falcatus; Boekelovia hooglandii; Borodinella; Botryococcus braunii, including B. sudeticus; Bracteoccocus, including B. aerius, B. grandis, B. cinnabarinas, B. minor, and B. medionucleatus; Carteria; Chaetoceros, including C. gracilis, C. muelleri, and C. muelleri subsalsum; Chlorococcum, including C. infusionum; Chlorogonium; Chroomonas; Chrysosphaera; Cricosphaera; Crypthecodinium cohnii; Cryptomonas; Cyclotella, including C. cryptica and C. meneghiniana; Desmodesmus; Dunaliella, including D. bardawil, D. bioculata, D. granulate, D. maritime, D. minuta, D. parva, D. peircei, D. primolecta, D. salina, D. terricola, D. tertiolecta, and D. viridis; Eremosphaera, including E. viridis; Ellipsoidon; Euglena; Franceia; Fragilaria, including F. crotonensis; Gleocapsa; Gloeothamnion; Hymenomonas; Isochrysis, including I. aff galbana and I. galbana; Lepocinclis; Micractinium (including UTEX LB 2614); Monoraphidium, including M. minutum; Monoraphidium; Nannochloris; Nannochloropsis, including N. salina; N. avicula, including N. acceptata, N. biskanterae, N. pseudotenelloides, N. pelliculosa, and N. saprophila; Neochloris oleabundans; Nephrochloris; Nephroselmis; Nitschia communis; Nitzschia, including N. alexandrine, N. communis, N. dissipata, N. frustulum, N. hantzschiana, N. inconspicua, N. intermedia, N. microcephala, N. pusilla, N. pusilla elliptica, N. pusilla monoensis, and N. quadrangular; Ochromonas; Oocystis, including O. parva and O. pusilla; Oscillatoria, including O. limnetica and O. subbrevis; Parachlorella, including P. beijerinckii (including strain SAG 2046) and P. kessleri (including any of SAG strains 11.80, 14.82, 21.11H9); Pascheria, including P. acidophila; Pavlova; Phagus; Phormidium; Platymonas; Pleurochrysis, including P. carterae and P. dentate; Prototheca, including P. stagnora (including UTEX 327), P. portoricensis, and P. moriformis (including UTEX strains 1441, 1435, 1436, 1437, 1439); Pseudochlorella aquatica; Pyramimonas; Pyrobotrys; Rhodococcus opacus; Sarcinoid chrysophyte; Scenedesmus, including S. armatus and S. rubescens; Schizochytrium; Spirogyra; Spirulina platensis; Stichococcus; Synechococcus; Tetraedron; Tetraselmis, including T. suecica; Thalassiosira weissflogii; and Viridiella fridericiana.

A suitable source of microalgae for the animal feed composition and animal feed supplement of the present invention is algal biomass. Algal biomass is material produced by growth and/or propagation of microalgal cells. Biomass may contain cells and/or intracellular contents as well as extracellular material. Extracellular material includes, but is not limited to, compounds secreted by a cell.

Typically, microalgae are cultured in liquid media to propagate biomass. For example, microalgal species may be grown in a medium containing a fixed carbon and/or fixed nitrogen source in the absence of light. Such growth is known as heterotrophic growth. For some species of microalgae, heterotrophic growth for extended periods of time such as 10 to 15 or more days under limited nitrogen conditions results in accumulation of high lipid content in the microalgal cells.

One particularly suitable source of microalgae for use in the present invention is microalgae cultivated for biofuel production. Microalgae cultivated for biofuel production includes algae before oils have been harvested from the algae (full-fat algae) and algae that has undergone oil extraction (defatted algae). Thus, as used herein, defatted algae has undergone an oil extraction process and so contains less oil relative to algae prior to oil extraction. Cells of defatted algae are predominantly lysed. Defatted algae include algal biomass that has been solvent (hexane) extracted.

Oils harvested from algae include any triacylglyceride (or triglyceride oil) produced by algae. Defatted algae contain less oil by dry weight or volume than the microalgae contained before extraction. In one embodiment, defatted algae include algae having 50-90% of its oil extracted so that the defatted algae contains, for example about 10-50% of the oil content of biomass before extraction. However, the biomass still has a high nutrient value in content of protein and other constituents which makes it suitable for use in animal feed.

The process of preparing defatted (or delipidated) algae for use in the animal feed composition and supplement of the present invention can be carried out by standard methods known to those of ordinary skill in the art. For example, algal cells can be lysed, which can be achieved by any convenient means, including heat-induced lysis, adding a base, adding an acid, using enzymes such as proteases and polysaccharide degradation enzymes such as amylases, using ultrasound, mechanical pressure-based lysis, and lysis using osmotic shock. Each of these methods for lysing a microorganism can be used as a single method or in combination simultaneously or sequentially. The extent of cell disruption can be observed by microscopic analysis. Using one or more of the methods above, typically more than 70% cell breakage is observed.

Lipids and oils generated by the microalgae can be recovered by extraction. In some cases, extraction can be performed using an organic solvent or an oil, or can be performed using a solventless-extraction procedure.

For organic solvent extraction of the microalgal oil, the preferred organic solvent is hexane. Typically, the organic solvent is added directly to the lysate without prior separation of the lysate components. In one embodiment, the lysate generated by one or more of the methods described above is contacted with an organic solvent for a period of time sufficient to allow the lipid components to form a solution with the organic solvent. In some cases, the solution can then be further refined to recover specific desired lipid components. The mixture can then be filtered and the hexane removed by, for example, rotoevaporation. Hexane extraction methods are well known in the art (see, e.g., Frenz et al., “Hydrocarbon Recovery by Extraction with a Biocompatible Solvent from Free and Immobilized Cultures of Botryococcus-braunii,” Enzyme Microb. Technol. 11:717-724 (1989), which is hereby incorporated by reference in its entirety.

Miao and Wu, “Biodiesel Production from Heterotrophic Microalgal Oil,” Biosource Technology 97:841-846 (2006), which is hereby incorporated by reference in its entirety, describe a protocol of the recovery of microalgal lipid from a culture of Chlorella protothecoides in which the cells were harvested by centrifugation, washed with distilled water, and dried by freeze drying. The resulting cell powder was pulverized in a mortar and then extracted with n-hexane.

In some cases, microalgal oils can be extracted using liquefaction (see, e.g., Sawayama et al., “Possibility of Renewable Energy Production and CO₂ Mitigation by Thermochemical Liquefaction of Microalgae,” Biomass and Bioenergy 17:33-39 (1999), which is hereby incorporated by reference in its entirety); oil liquefaction (see, e.g., Minowa et al., “Oil Production from Algal Cells of Dunaliella tertiolecta by Direct Thermochemical Liquefaction,” Fuel 74(12):1735-1738 (1995), which is hereby incorporated by reference in its entirety); or supercritical CO₂ extraction (see, e.g., Mendes et al., “Supercritical Carbon Dioxide Extraction of Compounds with Pharmaceutical Importance from Microalgae,” Inorganica Chimica Acta 356:328-334 (2003), which is hereby incorporated by reference in its entirety). Algal oil extracted via supercritical CO₂ extraction contains all of the sterols and carotenoids from the algal biomass and naturally do not contain phospholipids as a function of the extraction process. The residual from the processes essentially comprises defatted (or delipidated) algal biomass devoid of oil, but still retains the protein and carbohydrates of the pre-extraction algal biomass. Thus, the residual defatted algal biomass is a suitable source of protein concentrate/isolate and dietary fiber.

Oil extraction also includes the addition of an oil directly to a lysate without prior separation of the lysate components. After addition of the oil, the lysate separates either of its own accord or as a result of centrifugation or the like into different layers. The layers can include in order of decreasing density: a pellet of heavy solids, an aqueous phase, an emulsion phase, and an oil phase. The emulsion phase is an emulsion of lipids and aqueous phase. Depending on the percentage of oil added with respect to the lysate (w/w or v/v), the force of centrifugation, if any, volume of aqueous media, and other factors, either or both of the emulsion and oil phases can be present. Incubation or treatment of the cell lysate or the emulsion phase with the oil is performed for a time sufficient to allow the lipid produced by the microorganism to become solubilized in the oil to form a heterogeneous mixture.

Lipids can also be extracted from a lysate via a solventless extraction procedure without substantial or any use of organic solvents or oils by cooling the lysate. Sonication can also be used, particularly if the temperature is between room temperature and 65° C. Such a lysate on centrifugation or settling can be separated into layers, one of which is an aqueous:lipid layer. Other layers can include a solid pellet, an aqueous layer, and a lipid layer. Lipid can be extracted from the emulsion layer by freeze thawing or otherwise cooling the emulsion. In such methods, it is not necessary to add any organic solvent or oil. If any solvent or oil is added, it can be below 5% v/v or w/w of the lysate.

Algae used in the composition and feed supplement of the present invention is typically dried and/or ground into algal meal. Drying microalgal biomass, either predominantly intact or in homogenate form, is advantageous to facilitate further processing or for use of the biomass in the composition and feed supplement of the present invention. Drying refers to the removal of free or surface moisture/water from predominantly intact biomass or the removal of surface water from a slurry of homogenized (e.g., by micronization) biomass. In some cases, drying the biomass may facilitate a more efficient microalgal oil extraction process.

In one embodiment, concentrated microalgal biomass is drum dried to a flake form to produce algal flake. In another embodiment, the concentrated microalgal biomass is spray or flash dried (i.e., subjected to a pneumatic drying process) to form a powder containing predominantly intact cells to produce algal powder. In another embodiment, the concentrated microalgal biomass is micronized (homogenized) to form a homogenate of predominantly lysed cells that is then spray or flash dried to produce algal flour.

In certain embodiments, the algae component of the composition and/or feed supplement of the present invention is in the form of flour, flake, or powder and contains 15% or less, 10% or less, 5% or less, 2-6%, or 3-5% moisture by weight after drying.

The algae of the animal feed composition and/or animal feed supplement of the present invention may include only full-fat algae, only defatted (or delipidated) algae, or combinations thereof. When a full-fat algae is used in the animal feed composition, it may be desirable to reduce the amount of oil heterologous to the algae, particularly when more full-fat algae is present than defatted algae. Thus, for example, in one embodiment the animal feed composition includes more full-fat algae than defatted algae and the oil heterologous to the algae is present in the composition in an amount totaling 0.5-5% w/w of the composition. In another embodiment, the animal feed composition includes more defatted algae than full-fat algae and the oil heterologous to the algae is present in the composition in an amount totaling 3-15% w/w of the composition.

The algae component of the animal feed composition and animal feed supplement of the present invention may be substituted in the animal feed composition and animal feed supplement for another protein source having similar nutrient qualities to algae.

In one embodiment, the animal feed supplement of the present invention includes oil heterologous to the algae in an amount (w/w) of algae (1-25):oil (3-15).

In one embodiment of the animal feed composition and animal feed supplement, the oil heterologous to the algae comprises corn oil, although other types of oil may be used, including, without limitation, vegetable or seed oils derived from plants, including without limitation, oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, pium poppy, castor beans, pecan, jojoba, jatropha, macadamia, Brazil nuts, avocado, and combinations thereof. In one embodiment, the oil in the animal feed composition and/or supplement of the present invention is pure concentrated oil.

In the animal feed composition of the present invention, one or more grains are present in an amount totaling 50-70% w/w of the composition. Suitable grains include those commonly fed to animals, including, without limitation, maize, wheat, rice, sorghum, oats, potato, sweet potato, cassava, DDGS, and combinations thereof.

The animal feed composition of the present invention also includes a non-algal protein source in an amount totaling 15-30% w/w of the composition. Non-algal protein sources include those commonly part of animal feed, including, without limitation, meat, fish protein, soy protein, whey protein, wheat protein, bean protein, rice protein, pea protein, milk protein, etc. In one embodiment, the non-algal protein source is soybean, fishmeal, cottonseed meal, rapeseed meal, meat meal, plasma protein, blood meal, or combinations thereof.

In the animal feed composition of the present invention, an inorganic phosphate source is present in an amount totaling up to 1.5% w/w of the composition. High quality inorganic phosphates offer the combination of a consistently high total phosphorus content and excellent digestibility and are therefore widely used as supplemental phosphorus. Most inorganic phosphates used for this purpose are derived from natural rock phosphates, principally found in Africa, northern Europe, Asia, the Middle East and the United States. However, in their natural form these are unsuitable for direct use in animal feed because the phosphorus they contain cannot be metabolized by animals. Rock phosphates must therefore be chemically treated so that the phosphorus they contain is changed into the digestible orthophosphate form (PO4³—). During this process, close control of the production parameters is essential to avoid deterioration of the orthophosphate molecule into other unavailable forms of phosphorus, such as pyro- and meta-phosphate, and to ensure a suitable calcium to phosphorus ratio for animal nutrition. Furthermore, rock phosphates also contain impurities, such a fluorine, cadmium, and arsenic which, if not removed in the production process, make them unsuitable for animal nutrition. In one embodiment, the phosphate source in the animal feed composition of the present invention comprises dicalcium phosphate.

The animal feed composition of the present invention may further include any one or more of the following: plasma protein in an amount totaling 0.5-3.0% w/w of the composition; an inorganic calcium source in an amount totaling 0.1-10% w/w of the composition; a vitamin/mineral mix in an amount totaling 0.1-1% w/w of the composition, where the vitamin/mineral mix comprises one or more trace minerals; an inorganic magnesium source in an amount totaling 0.01-0.1% w/w of the composition; and an antibiotic in an amount totaling 0.01-0.1% w/w of the composition.

Similarly, the animal feed supplement of the present invention may further include one or more of the following: plasma protein in an amount (w/w) of algae (1-25):plasma protein (1-5); an inorganic calcium source in an amount (w/w) of algae (1-25):calcium (1-4); a vitamin/mineral mix comprising trace minerals, wherein the vitamin/mineral mix is provided in an amount (w/w) of algae (1-25):vitamin/mineral mix (0.1-2); an inorganic magnesium source in an amount (w/w) of algae (1-25):magnesium (0.01-0.1); and an antibiotic in an amount (w/w) of algae (1-25):antibiotic (0.01-0.1).

The animal feed composition and animal feed supplement of the present invention may also include added enzymes known to be useful as animal feed additives.

Plasma protein is a known supplement in animal feed, and has been used, e.g., to increase weight gain and feed efficiency of young pigs. Plasma protein is obtained by collecting blood from animals, preferably pigs or cows. For example, blood is collected at slaughter plants. As it is collected, the blood is held in a circulating stainless steel tank with anticoagulants such as sodium citrate or sodium phosphate to avoid clotting. The whole blood is then separated, likely by centrifugation into two parts, cellular material (red corpuscles, white corpuscles, and platelets) and plasma. Plasma is composed of about 60% albumin and about 40% globulin. After separation, the plasma is cooled in an insulated tanker until ready to dry.

The plasma component is then further concentrated 2 to 3 fold by membrane filtration. At this stage, a microbial fermentation extract containing primarily amylase may be added. Thus, in one embodiment, animal plasma protein may be combined with a microbial fermentation product with a significant level of amylase activity (see U.S. Pat. No. 5,372,811 to Yoder, which is hereby incorporated by reference in its entirety). The mixture is blended for 10 minutes and finally is co-dried to form a beige powdery substance. Spray drying should occur at temperatures low enough to maintain the highly digestible proteins but high enough to purify the dry powder eliminating bacterial and viral contamination.

Spray dried animal plasma protein has traditionally been used as a high quality protein used as a replacement for milk proteins due to its high quality protein and immunoglobulin content. This plasma has also been used in the feed industry as a feed supplement ingredient for veal and calf milk replacers, aquaculture, and pet food for its influence on voluntary feed intake and efficient gains equal to or better than milk proteins. In one embodiment, the animal plasma protein used in the animal feed and feed supplement compositions of the present invention is comprised of high levels of amino acids.

Inorganic calcium sources for use in the composition and animal feed supplement of the present invention are well known. In one embodiment, the inorganic calcium source is limestone (calcium carbonate). In other embodiments, the inorganic calcium source is from one or more of the three supplemental sources of inorganic calcium of calcite flour, aragonite, and albacar (see Wohlt et al., “Calcium Sources for Milk Production in Holstein Cows via Changes in Dry Matter Intake, Mineral Utilization, and Mineral Source Buffering Potential,” J. Dairy Sci. 70:2812 (1987), which is hereby incorporated by reference in its entirety). Each of these inorganic calcium sources differs in particle size and rate of reactivity.

Suitable vitamin/mineral mix for use in the animal feed composition and/or supplement of the present invention may include, for example, vitamin A, vitamin D₃, vitamin E, vitamin K, biotin, choline, choline chloride, folacin, folic acid, niacin, pantothenic acid, d-calcium pantothenate, pyridoxine hydrochloride, nicotinic acid, cyanocobalamin, riboflavin, thiamin, thiamine hydrochloride, menadione sodium bisulfite, ethoxyquin, vitamin B₆, vitamin B₁₂, Cu, I, Mn, Zn, Se, Mg, Co, or Fe, and combinations thereof.

Magnesium oxide (MgO) is a widely available inorganic magnesium source, in many different forms for different uses. Generally, an MgO intended for animal feed use is preferred, and a number of commercial suppliers are available.

Suitable antibiotics for the animal feed composition and supplement of the present invention may include, for example, tetracyclines, bacitracin, avilamycin, nicarbazin, tylosin (as tylosin phosphate), tiamulin, lincomycin, virginiamycin, quinolone antibacterials, carbadox, chlortetracycline hydrochloride, and combinations thereof.

In addition to one or more of the amino acids lysine, threonine, isoleucine, tryptophan, and methionine, other amino acids may be included in the animal feed composition and/or animal feed supplement of the present invention.

A further aspect of the present invention relates to a method of feeding an animal. This method involves administering to an animal the animal feed composition of the present invention. Yet another aspect of the present invention involves administering to an animal an animal feed in combination with the animal feed supplement of the present invention.

Algae, either full fat or defatted, contains high quality proteins, carbohydrates, fiber, ash, and other nutrients appropriate for animal feed. Animals that may be fed with the animal feed composition and/or animal feed supplement of the present invention include, without limitation, a ruminant, poultry, swine, aquaculture, pet, dog, cat, horse, zoo animal, mouse, rat, rabbit, guinea pig, and hamster. In one embodiment, the animal is a laying hen, a broiler chicken, or a weanling pig.

In one embodiment, the animal is a laying hen and the animal feed composition comprises corn or corn and soybean and either (i) defatted algae in an amount of about 7.5% w/w or (ii) defatted algae in an amount of about 15% w/w.

In another embodiment, the animal is a broiler chicken and the animal feed composition comprises corn, soybean, and either (i) defatted algae in an amount of about 7.5% w/w or (ii) defatted algae in an amount of about 7.5% w/w with the amino acid supplements Methionine, Lysine, Isoleucine, Threonine, Tryptophan, and Valine.

In a further embodiment, the animal is a pig or a weanling pig and the animal feed composition comprises corn, soybean, and either (i) defatted algae in an amount of about 7.5% w/w or (ii) full-fat algae in an amount of about 10% w/w with a fumaric acid supplement of about 2% w/w.

Yet a further aspect of the present invention relates to a method of improving the feed efficiency of an animal. This method involves administering to an animal an animal feed in combination with the animal feed supplement of the present invention under conditions effective to cause a 3-15% decrease in plasma uric acid levels in the animal relative to such animal receiving the animal feed without the animal feed supplement, thereby improving the feed efficiency in the animal.

Still another aspect of the present invention relates to a method of improving the feed efficiency of an animal. This method involves administering to an animal the animal feed composition of the present invention under conditions effective to cause a 3-15% decrease in plasma uric acid levels in the animal relative to such animal receiving an animal feed other than the animal feed composition, thereby improving the feed efficiency in the animal.

Another aspect of the present invention relates to an animal feed, where the improvement comprises algae in an amount effective to decrease uric acid levels in plasma in an animal by 3-15% after consuming the animal feed, thereby improving feed efficiency in the animal.

### EXAMPLES

### Example 1

### Microalgal Biomass Diet Supplementation Defatted and Full-fat Diatom Microalgal Biomass can Partially Replace Corn and Soybean Meal in the Diets of Weanling Pigs

Materials and Methods for Example 1

Animals and Dietary Treatments

The animal experiments were approved by the Institutional Animal Care and Use Committee of Cornell University. All pigs were weanling crossbreds (Yorkshire-Landrace-Hampshire) selected from the Cornell University Swine Farm. The pigs were weaned at 4 weeks of age, and allotted into treatment groups based on body weight, litter, and sex. The pigs were housed individually in pens (1×2.5 m) with concrete floors in a temperature-controlled barn (22-25° C.) with a light:dark cycle of 12:12 h. In both experiments, the pigs were adjusted for 4 days to a corn-soybean meal basal diet (“BD”) (BD1, BD2). All pigs had free access to feed and water, and were monitored daily.

A preliminary experiment was conducted to determine appropriate inclusion rates of full-fat algae (“FFA”) and de-fatted algae (“DFA”) in the experimental diets for replacing corn and soybean meal (“SBM”) in the diets of weanling pigs. Accordingly, 32 weanling pigs (13.4±1.6 kg body weight (“BW”)) were selected in Experiment 1 and divided into 4 groups (n=8/group) and fed BD1 (Table 1), BD1+7.5% DFA replacing SBM (“7.5% DFA-A”, Table 2), BD1+7.5% DFA replacing a combination of corn and SBM (“7.5% DFA-B”, Table 2), or BD1+15% DFA replacing a combination of corn and SBM (“15% DFA”, Table 2) for 6 weeks. The compositions of the basal diets are shown in Table 1. The compositions of defatted diatom algae diets used in Experiment 1 are shown in Table 2.

TABLE 1
Composition of Basal Diets (As-fed Basis)

Treatment

| | | |
|---|---|---|
| Item | BD1¹ | BD2² |

Ingredient, %

| | | |
|---|---|---|
| Corn | 65.60 | 67.24 |
| Soybean meal (48% CP) | 28.00 | 26.80 |
| Corn oil | 1.00 | 1.00 |
| De-fatted diatom algae³ | — | — |
| Full-fat diatom algae³ | — | — |
| Plasma, spray-dried | 1.50 | 1.50 |
| Limestone | 1.05 | 0.90 |
| Dicalcium phosphate | 1.50 | 0.99 |
| Vitamin/mineral premix⁴ | 0.20 | 0.50 |
| Salt | 0.50 | 0.50 |
| Magnesium oxide | 0.05 | 0.05 |
| DL-Met | — | 0.01 |
| L-Lys-HCL | 0.10 | — |
| L-Threonine | — | 0.01 |
| Antibiotic⁵ | 0.50 | 0.50 |

Calculated nutrient composition

| | | |
|---|---|---|
| ME,⁶ kcal/kg | 3,274 | 3,151 |
| CP, % | 19.9 | 17.8 |
| CF, % | 4.43 | 4.67 |
| Ca, % | 0.84 | 0.89 |
| Total P, % | 0.68 | 0.69 |
| Lys, % | 1.20 | 0.96 |
| TSAA, % | 0.68 | 0.61 |
| Thr, % | 0.78 | 0.70 |
| Trp, % | 0.24 | 0.19 |

¹BD1 = Corn-soybean meal basal diet in Experiment 1.
²BD2 = Corn-soybean meal basal diet in Experiment 2.
³Nutrient compositions of defatted and full-fat diatom algae were analyzed by Dairy One, Inc., Ithaca, NY 14850, and Experiment Station Chemical Laboratories, University of Missouri.
⁴Vitamin and mineral premix supplied the following amounts (per kilogram of diet): Vitamin A, 2,200 IU; vitamin D₃, 220 IU; vitamin E, 16 IU; vitamin K, 0.5 mg; biotin, 0.05 mg; choline, 0.5 g; folacin, 0.3 mg; niacin, 15 mg; pantothenic acid, 10 mg; riboflavin, 3.5 mg; thiamin, 1 mg; vitamin B₆, 1.5 mg; vitamin B₁₂, 17.5 μg; Cu, 6 mg; I, 0.14 mg; Mn, 4 mg; Zn 100 mg; Se, 0.3 mg; Mg, 0.4 mg; Fe, 80 mg.
⁵Antibiotic additive for BD1 (Tylan ® 10) contained tylosin (as tylosin phosphate) at 22 g/kg (Elanco). BD2 contained 55 mg of chlortetracycline hydrochloride.
⁶Calculated based on NRC (1998).

TABLE 2
Composition of Defatted Diatom Algae Diets in Experiment 1

| | Treatment | | |
|---|---|---|---|
| Item | 7.5% DFA- A¹ | 7.5% DFA- B² | 15% DFA³ |

Ingredient, %

| | | | |
|---|---|---|---|
| Corn | 65.76 | 63.9 | 60.83 |
| Soybean meal (48% CP) | 20.5 | 22.9 | 19 |
| Corn oil | 1 | 1 | 1 |
| De-fatted diatom algae² | 7.5 | 7.5 | 15 |
| Plasma, spray-dried | 1.5 | 1.5 | 1.5 |
| Limestone | 0.7 | 0.65 | 0.4 |
| Dicalcium phosphate | 1.5 | 1.3 | 1 |
| Vitamin/mineral premix³ | 0.2 | 0.2 | 0.2 |
| Salt | 0.5 | 0.3 | 0.3 |
| Magnesium oxide | 0.05 | 0.05 | 0.05 |
| L-Lys-HCL | 0.25 | 0.2 | 0.2 |
| L-Threonine | 0.04 | — | 0.02 |
| Antibiotic⁴ | 0.5 | 0.5 | 0.5 |

Calculated nutrient composition

| | | | |
|---|---|---|---|
| ME,⁵ kcal/kg | 3,274 | 3,151 | 3,158 |
| CP, % | 19.9 | 17.8 | 18.8 |
| CF, % | 4.43 | 4.67 | 4.75 |
| Ca, % | 0.84 | 0.89 | 0.84 |
| Total P, % | 0.68 | 0.69 | 0.66 |
| Lys, % | 1.20 | 0.96 | 1.19 |
| TSAA, % | 0.68 | 0.61 | 0.64 |
| Thr, % | 0.78 | 0.70 | 0.74 |
| Trp, % | 0.24 | 0.19 | 0.21 |

¹7.5% DFA-A = 7.5% soybean meal replaced with defatted algal biomass.
²7.5% DFA-B = 7.5% combination of corn and soybean meal replaced with defatted algal biomass.
³15% DFA = 15% combination of corn and soybean meal replaced with defatted algal biomass.

In Experiment 2, 40 weanling pigs (9.6±0.8 kg BW) were divided into 4 groups (n=10/group) and fed BD2 (Table 1), BD2+10% FFA replacing a combination of corn and SBM (“10% FFA”, Table 3), BD2+10% FFA+2% fumaric acid (Univar, Morrisville, Pa.) (“10% FFA+FA”, Table 3), or BD2+10% FFA+50% higher levels of Cu, Se, and Zn than in the premix of BD2 (“10% FFA+TM”, Table 3) for 6 weeks (Table 4). The compositions of full fat diatom algae diets used in Experiment 2 are shown in Table 3.

TABLE 3
Composition of Full Fat Diatom Algae Diets in Experiment 2

| | Treatment | | |
|---|---|---|---|
| | | 10% FFA + | 10% FFA + |
| Item | 10% FFA¹ | FA² | TM³ |

Ingredient, %

| | | | |
|---|---|---|---|
| Corn | 64.57 | 62.87 | 64.77 |
| Soybean meal (48% CP) | 19.3 | 19.3 | 19.3 |
| Corn oil | 2 | 2 | 2 |
| De-fatted diatom algae | — | — | — |
| Full-fat diatom algae² | 10 | 10 | 10 |
| Plasma, spray-dried | 1.5 | 1.5 | 1.5 |
| Limestone | 0.97 | 0.97 | 0.97 |
| Dicalcium phosphate | 0.5 | 0.5 | 0.5 |
| Vitamin/mineral premix³ | 0.3 | — | — |
| Salt | 0.09 | 0.09 | 0.09 |
| Magnesium oxide | 0.05 | 0.05 | 0.05 |
| L-Lys-HCL | 0.09 | 0.09 | 0.09 |
| L-Threonine | 0.09 | 0.09 | 0.09 |
| Antibiotic⁴ | 0.04 | 0.04 | 0.04 |

Calculated nutrient composition

| | | | |
|---|---|---|---|
| ME,⁵ kcal/kg | 3,180 | 3,114 | 3,183 |
| CP, % | 17.1 | 16.9 | 17.1 |
| CF, % | 6.06 | 5.98 | 6.06 |
| Ca, % | 0.68 | 0.68 | 0.68 |
| Total P, % | 0.58 | 0.58 | 0.58 |
| Lys, % | 0.98 | 0.97 | 0.97 |
| TSAA, % | 0.68 | 0.68 | 0.68 |
| Thr, % | 0.74 | 0.74 | 0.74 |
| Trp, % | 0.22 | 0.22 | 0.22 |

¹10% FFA = 10% corn and soybean meal replaced with full-fat algae.
²10% FFA + FA = 10% corn and soybean meal replaced with full-fat algae, and supplemented with 2% fumaric acid.
³10% FFA + TM = 10% corn and soybean meal replaced with full fat algae, and supplemented with 50% higher levels of trace minerals Cu, Se, and Zn than in the BD2 premix.

TABLE 4
Analyzed Mineral Concentration of Diets in Experiment 2

| | Treatment | | | |
|---|---|---|---|---|
| Item | BD1¹ | 7.5% DFA-A² | 7.5% DFA-B³ | 15% DFA⁴ |

Macromineral, g/kg

| | | | | |
|---|---|---|---|---|
| Ca | 6.1 | 6.2 | 6.0 | 8.6 |
| K | 6.8 | 7.3 | 7.7 | 9.4 |
| Mg | 1.6 | 2.4 | 2.3 | 3.1 |
| Na | 2.2 | 3.0 | 3.0 | 6.7 |
| P | 6.2 | 7.4 | 6.8 | 6.8 |
| S | 1.9 | 2.4 | 2.4 | 3.2 |

Micromineral, mg/kg

| | | | | |
|---|---|---|---|---|
| Al | 132.5 | 197.6 | 193.3 | 205.4 |
| As | 7.9 | 7.8 | 5.4 | 9.9 |
| B | 227.7 | 243.1 | 227.7 | 245.3 |
| Ba | 0.7 | 1.3 | 0.9 | 1.0 |
| Ca | 0.3 | 0.1 | 0.1 | 0.1 |
| Co | 0.5 | 0.9 | 1.8 | 1.2 |
| Cr | 4.7 | 7.7 | 8.7 | 9.7 |
| Cu | 15.3 | 13.6 | 15.4 | 22.5 |
| Fe | 224.8 | 394.6 | 386.9 | 531.0 |
| Mn | 37.1 | 49.3 | 63.7 | 58.2 |
| Mo | — | — | — | — |
| Ni | 2.4 | 2.3 | 4.7 | 4.2 |
| Pb | 11.3 | 7.2 | 4.5 | 6.2 |
| Se | 0.1 | 0.1 | 0.1 | 0.1 |
| Si | 89.9 | 941.8 | 926.5 | 1205.9 |
| Sr | 5.7 | 68.4 | 37.0 | 108.0 |
| Ti | 2.7 | 6.3 | 8.2 | 11.5 |
| Zn | 118.1 | 99.4 | 91.3 | 119.9 |

¹BD1 = Corn-soybean meal basal diet in Experiment 1.
²7.5% DFA-A = 7.5% soybean meal replaced with defatted algal biomass.
³7.5% DFA-B = 7.5% combination of corn and soybean meal replaced with defatted algal biomass.
⁴15% DFA = 15% combination of corn and soybean meal replaced with defatted algal biomass.

biomass.

The selection and level of Cu, Se, and Zn in the 10% FFA+TM were based on fecal and plasma mineral analyses from Experiment 1. Samples of FFA and DFA were subjected to proximate analysis and amino acid profiling (Table 5); and all experimental diets were assayed for mineral concentrations (Tables 6 and 7).

TABLE 5
Proximate and Amino Acid Composition of Defatted
and Full Fat Diatom Algae Products (as fed basis)

| | | |
|---|---|---|
| Item | DFA¹ | FFA² |

Proximate composition³, %

| | | |
|---|---|---|
| Moisture | 6.9 | 14.2 |
| DM | 93.1 | 85.8 |
| CP | 19.1 | 13.9 |
| CF | 3.3 | 9.3 |
| ADF | 0.7 | 2.3 |
| NDF | 1.4 | 16 |
| TDN | 37 | 34 |
| Ash | 44.5 | 39.95 |

Total amino acid content⁴, %

| | | |
|---|---|---|
| Aspartic acid | 1.88 | 1.31 |
| Threonine | 0.88 | 0.63 |
| Serine | 0.76 | 0.53 |
| Glutamic acid | 1.81 | 1.29 |
| Proline | 0.65 | 0.45 |
| Glycine | 0.96 | 0.67 |
| Alanine | 1.09 | 0.76 |
| Cysteine | 0.32 | 0.19 |
| Valine | 0.98 | 0.7 |
| Methionine | 0.33 | 0.26 |
| Isoleucine | 0.78 | 0.55 |
| Leucine | 1.33 | 0.94 |
| Tyrosine | 0.57 | 0.4 |
| Phenylalanine | 0.86 | 0.61 |
| Lysine | 0.83 | 0.57 |
| Histidine | 0.3 | 0.18 |
| Arginine | 0.93 | 0.61 |
| Tryptophan | 0.18 | 0.12 |

¹DFA = De-fatted diatom algae biomass.
²FFA = Full-fat diatom algae.
³Samples were analyzed by Dairy One, Inc., Ithaca, NY 14850.
⁴Samples were analyzed in Experiment Station Chemical Laboratories, University of Missouri.

TABLE 6
Analyzed Mineral Concentration of Diets in Experiment 2

Treatment

| | | | | |
|---|---|---|---|---|
| | | | 10% FFA + | 10% FFA + |
| Item | BD2¹ | 10% FFA² | FA³ | TM⁴ |

Macromineral, g/kg

| | | | | |
|---|---|---|---|---|
| Ca | 6.7 | 8.5 | 5.4 | 6.8 |
| K | 10.4 | 10.2 | 10.4 | 9.9 |
| Mg | 1.8 | 2.1 | 2.3 | 2.0 |
| Na | 2.9 | 5.3 | 3.7 | 3.1 |
| P | 6.3 | 5.6 | 6.2 | 5.4 |
| S | 2.0 | 2.4 | 2.4 | 2.2 |

Micromineral, mg/kg

| | | | | |
|---|---|---|---|---|
| Al | 137.0 | 98.0 | 106.2 | 94.2 |
| As | 0.2 | — | 0.2 | — |
| B | 71.2 | 60.9 | 64.4 | 70.2 |
| Ba | 1.2 | 1.2 | 1.0 | 1.4 |
| Ca | 0.1 | 0.1 | 0.1 | 0.1 |
| Co | 0.1 | 0.2 | 0.2 | 0.2 |
| Cr | 1.4 | 1.2 | 1.3 | 1.2 |
| Cu | 12.6 | 10.5 | 12.2 | 11.4 |
| Fe | 212.1 | 268.1 | 280.8 | 240.8 |
| Mn | 27.7 | 25.2 | 28.7 | 28.8 |
| Mo | 0.7 | 0.3 | 0.5 | 0.3 |
| Ni | 1.8 | 1.7 | 1.9 | 1.9 |
| Pb | 0.4 | 1.0 | 0.9 | 0.9 |
| Se | 0.5 | 0.5 | 0.4 | 0.6 |
| Si | 17.7 | 25.0 | 22.0 | 20.6 |
| Sr | 4.1 | 116.3 | 56.3 | 87.2 |
| Ti | 3.0 | 3.5 | 3.8 | 3.2 |
| Zn | 142.6 | 116.3 | 114.0 | 141.8 |

¹BD2 = Corn-soybean meal basal diet in Experiment 2.
²10% FFA = 10% corn and soybean meal replaced with full-fat algae.
³10% FFA + FA = 10% corn and soybean meal replaced with full-fat algae, and supplemented with 2% fumaric acid.
⁴10% FFA + TM = 10% corn and soybean meal replaced with full-fat algae, and supplemented with 50% higher levels of trace minerals Cu, Se, and Zn than in the BD2 premix.

TABLE 7
Effect of Dietary Defatted Diatom Microalgal Biomass
on Overall Growth Performance of Pigs in Experiment 1

| | Treatment | | | | | | | |
|---|---|---|---|---|---|---|---|---|
| | | 7.5% | 7.5% | | | Main effect, P-value | | |
| | | DFA- | DFA- | 15% | | | | Diet × |
| Item | BD1¹ | A² | B³ | DFA⁴ | SEM | Diet | Week | Week |
| ADG, | 0.88^{a} | 0.78^{b} | 0.82^{ab} | 0.78^{b} | 0.02 | 0.02 | <0.0001 | 0.70 |
| kg | | | | | | | | |
| ADFI, | 1.47 | 1.47 | 1.50 | 1.46 | 0.05 | 0.92 | <0.0001 | 0.99 |
| kg | | | | | | | | |
| G:F, | 0.60^{a} | 0.55^{b} | 0.59^{a} | 0.53^{b} | 0.02 | 0.045 | <0.0001 | 0.68 |
| kg/kg | | | | | | | | |

^{a,b}Within a row, means without a common superscript, differ (P < 0.05).
¹BD1 = Corn-soybean meal basal diet in Experiment 1.
²7.5% DFA-A = 7.5% soybean meal replaced with defatted algal biomass.
³7.5% DFA-B = 7.5% combination of corn and soybean meal replaced with defatted algal biomass.
⁴15% DFA = 15% combination of corn and soybean meal replaced with defatted algal biomass.

Growth Performance and Sample Collection

Orts from individual pigs were collected daily, and BW and feed intake (“FI”) were recorded biweekly to calculate average daily feed intake (“ADFI”), average daily gain (“ADG”), and gain:feed (“G:F”). Whole blood samples of all individual pigs were collected initially and then biweekly from the anterior vena cava using heparinized tubes (158 USP units; Vacutainer, Becton Dickinson, Franklin Lakes, N.Y.) after an overnight fast (8 hours) for assays of blood hemoglobin content and packed cell volume in Experiment 2. The collected whole blood samples were chilled on ice and centrifuged at 3000×g for 10 minutes at 4° C. (GS-6KR centrifuge, Beckman Instruments, Palo Alto, Calif.) to prepare plasma samples for biochemical assays. In Experiment 1, fresh fecal samples from each individual pig were collected using sterile utensils at the end of study (week 6), placed in pre-weighed sterile 50-mL conical tubes, and stored on ice for transport to the laboratory. The wet weights of the fecal samples were determined, and the samples were stored at −80° C. until freeze-drying.

Body Lean Yield Predictions

In Experiment 1, each pig was subject to an ultrasound scan using an Aloka 5011 probe (Model 500V B mode scanner; Corometrics Medical Systems, Wallingford, Conn.) at the end of study. Animals were restrained in ventral recumbency, and the image was taken after aligning the last rib on the ultrasound grid, then guiding the probe along the midline until reaching the intercostal muscle lining. Automatic measurements of the vertebral fat and loin depths were taken, and percentage body lean yield was predicted using the AUSKey automated measuring system (Animal Ultrasound Services, AUSkey System, Ithaca, N.Y.).

Fecal Dry Matter and Mineral Analyses

Frozen fecal samples collected from Experiment 1 were placed in a lyophilizer (Freeze-dry Specialities, Osseo, Minn.) for 36 hours, weighed to calculate the percentage of dry matter, and stored at −20° C. For mineral analyses, freeze-dried fecal samples (100 g) were thawed at room temperature and ground to pass through a 1 mm screen, and plasma samples (200 μL) were thawed at 4° C. The concentrations of individual elements in both the fecal and plasma samples were measured using an inductively couple argon plasma spectrophotometer (ICAP 61E Trace Analyzer, Thermo Jarrell Ash corporation, Franklin, Mass.). Before analysis, samples were digested in a mixture of HNO₃ and HClO₄ (9:1, volume/volume), and diluted in 5% HNO₃. Standard reference materials (No. 1573a, tomato leaves, and No. 1577b, bovine liver, National Institute of Standards and Technology, Gaithersburg, Md.) were used to validate the analytical procedures (House et al., “Mineral Accretion in the Fetus and Adnexa During Late Gestation in Holstein Cows,” J. Dairy Sci. 76:2999-3010 (1993), which is hereby incorporated by reference in its entirety).

Plasma and Blood Biochemical Analyses

Plasma alkaline phosphatase activity was measured by the hydrolysis of p-nitrophenol phosphate to p-nitrophenol (Bowers et al., “A Continuous Spectrophotometric Method for Measuring the Activity of Serum Alkaline Phosphatase,” Clin. Chem.12:70-89 (1966), which is hereby incorporated by reference in its entirety). The enzyme unit was defined as 1 μmol of p-nitrophenol released per minute at 30° C. Plasma alanine transaminase activity was measured using a kit as described by the manufacturer's instructions (Thermo Scientific, Waltham, Mass.). Plasma urea nitrogen concentration was determined by modified methods described previously (Fawcett et al., “A Rapid and Precise Method for the Determination of Urea,” J. Clin. Pathol. 13:156-159 (1960) and Chaney et al., “Modified Reagents for Analysis of Urea and Ammonia,” Clin. Chem. 8:130-132 (1962), which are hereby incorporated by reference in their entirety). Total triglyceride, cholesterol, and non-esterified fatty acids were analyzed using enzymatic colorimetric kits (Wako L-Type Triglyceride M, Cholesterol E, and NEFA C, respectively). Plasma inorganic phosphorus concentration was determined using Elon (p-methyl-aminophenol sulfate) solution after de-proteination with 12.5% trichloroacetic acid (Gomori, G., “A Modification of the Colorimetric Phosphorus Determination for Use with the Photoelectric Colorimeter,” J. Lab. Clin. Med. 27:955-960 (1942), which is hereby incorporated by reference in its entirety). Packed cell volume was determined after whole-blood was drawn into heparinized microcapillary tubes (Fisher Scientific, Pittsburgh, Pa.), sealed, and centrifuged at 2000×g for 12 min. Blood hemoglobin concentrations were measured spectrophotometrically using the cyanomethemoglobin method, following the manufacturer's instructions (Pointe Scientific, Canton, Mich.).

Statistical Analyses

Data were analyzed using the General Linear Models procedure of SAS (SAS Inst. Inc, Cary, N.C.). In both experiments, the main effects of dietary treatments on growth performance and plasma biochemical measures were subjected to one-way ANOVA with time-repeated measurements (Gill, J. L., “Repeated Measurement: Sensitive Tests for Experiments with Few Animals,” J. Anim. Sci. 63:943-954 (1986), which is hereby incorporated by reference in its entirety). Data of other measures were analyzed using one-way ANOVA. Duncan's multiple range test was used to compare treatment means. For all analyses, pooled SEM were listed and the significance level for differences was P<0.05.

Results for Example 1

Experiment 1

Compared with pigs fed only BD1, those fed 7.5% DFA-A or 15% DFA had lower (P<0.05) overall ADG (by 11%) and G:F (by 9 and 11%, respectively; Table 7). Pigs fed 7.5% DFA-B had lower (P<0.05) overall G:F (by 8%) than pigs fed BD1. All 4 dietary treatment groups of pigs had similar biweekly or overall ADFI, plasma alkaline phosphatase activities, plasma alanine aminotransferase activities, plasma urea nitrogen concentrations, and plasma lipid profiles of total cholesterol, total triglycerides, and total non-esterified fatty acid concentrations (Table 8).

TABLE 8
Effect of Dietary Defatted Diatom Microalgal Biomass on
Plasma Biochemical Measures of Pigs in Experiment 1

| | | Treatment | | | | | Main effect, P-value | | |
|---|---|---|---|---|---|---|---|---|---|
| | | | 7.5% | 7.5% | 15% | | | | Diet × |
| Item | Week | BD1¹ | DFA-A² | DFA-B³ | DFA⁴ | SEM | Diet | Week | Week |
| Alkaline | 0 | 81.0 | 72.1 | 77.5 | 83.1 | 4.0 | 0.97 | 0.03 | 0.99 |
| phosphatase | 6 | 80.9 | 78.0 | 80.4 | 85.8 | 4.1 | | | |
| activity, U/L⁵ | | | | | | | | | |
| Alanine | 0 | 19.0 | 19.1 | 22.9 | 22.6 | 1.4 | 0.94 | <0.001 | 0.98 |
| aminotransferase | 6 | 18.9 | 17.2 | 19.7 | 20.8 | 1.2 | | | |
| activity, U/L | | | | | | | | | |
| Urea N, mg/dL | 0 | 10.0 | 9.9 | 9.9 | 10.0 | 0.5 | 0.13 | <0.001 | 0.78 |
| | 6 | 15.2 | 14.2 | 14.7 | 15.0 | 0.7 | | | |
| Total | 0 | 64.2 | 56.7 | 56.3 | 58.6 | 5.5 | 0.51 | <0.0001 | 0.83 |
| cholesterol, | 6 | 107.4 | 102.9 | 105.0 | 97.9 | 5.0 | | | |
| mg/dL | | | | | | | | | |
| Total | 0 | 44.2 | 49.0 | 49.3 | 52.4 | 5.1 | 0.98 | <0.0001 | 0.16 |
| triglyceride, | 6 | 39.4 | 33.6 | 30.8 | 29.3 | 2.9 | | | |
| mg/dL | | | | | | | | | |
| Total non- | 0 | 48.8 | 59.7 | 70.7 | 50.5 | 14.4 | 0.82 | 0.003 | 0.95 |
| esterified fatty | 6 | 95.9 | 101.1 | 97.7 | 88.9 | 19.8 | | | |
| acid, μmol/L | | | | | | | | | |

¹BD1 = Corn-soybean meal basal diet in Experiment 1.
²7.5% DFA-A = 7.5% soybean meal replaced with defatted algal biomass.
³7.5% DFA-B = 7.5% combination of corn and soybean meal replaced with defatted algal biomass.
⁴15% DFA = 15% combination of corn and soybean meal replaced with defatted algal biomass.
⁵The enzyme unit was defined as the amount of activity that releases 1 μmol of p-nitrophenol per minute at 30° C.

Additionally, no significant differences were detected in the body lean yield predictions among all 4 treatment groups (Table 9).

TABLE 9
Effect of Dietary De-fatted Diatom Microalgal Biomass
on Predicted Lean Yield of Pigs in Experiment 1

| | Treatment | | | | | |
|---|---|---|---|---|---|---|
| | | 7.5% | 7.5% | 15% | | |
| Item | BD1¹ | DFA-A² | DFA-B³ | DFA⁴ | SEM | P-value |
| aPLean | 53.1 | 52.9 | 53.2 | 52.4 | 0.42 | 0.37 |

¹BD1 = Corn-soybean meal basal diet in Experiment 1.
²7.5% DFA-A = 7.5% soybean meal replaced with defatted algal biomass.
³7.5% DFA-B = 7.5% combination of corn and soybean meal replaced with defatted algal biomass.
⁴15% DFA = 15% combination of corn and soybean meal replaced with defatted algal biomass.

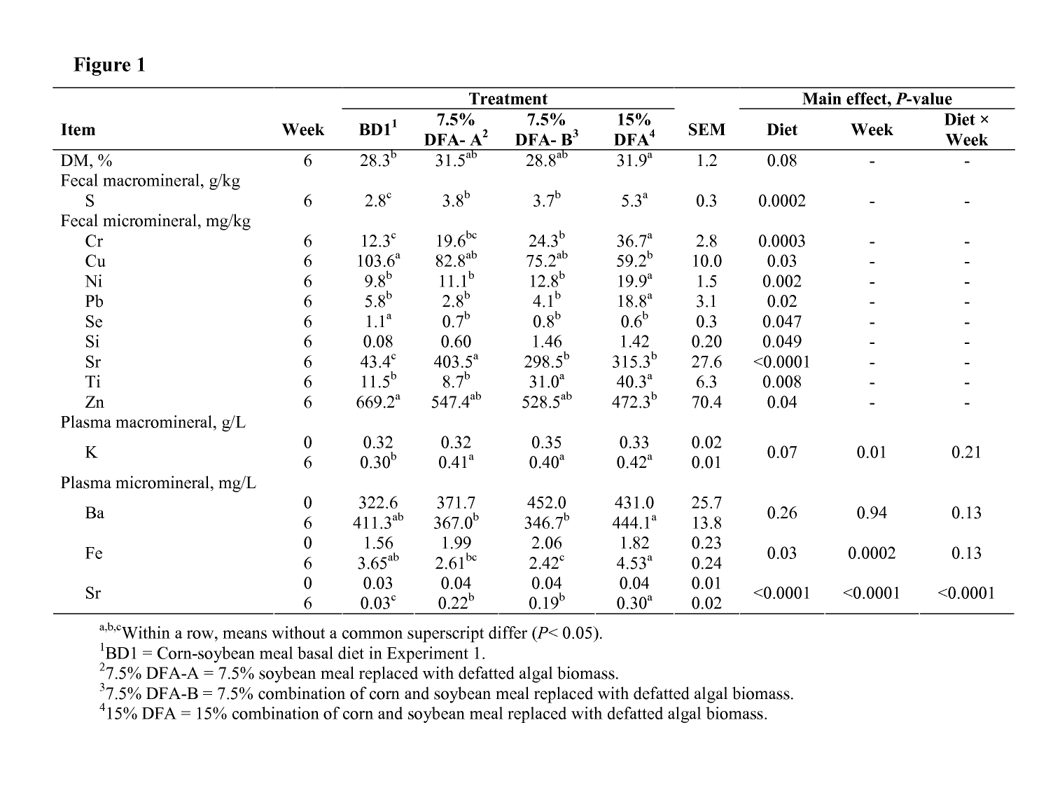

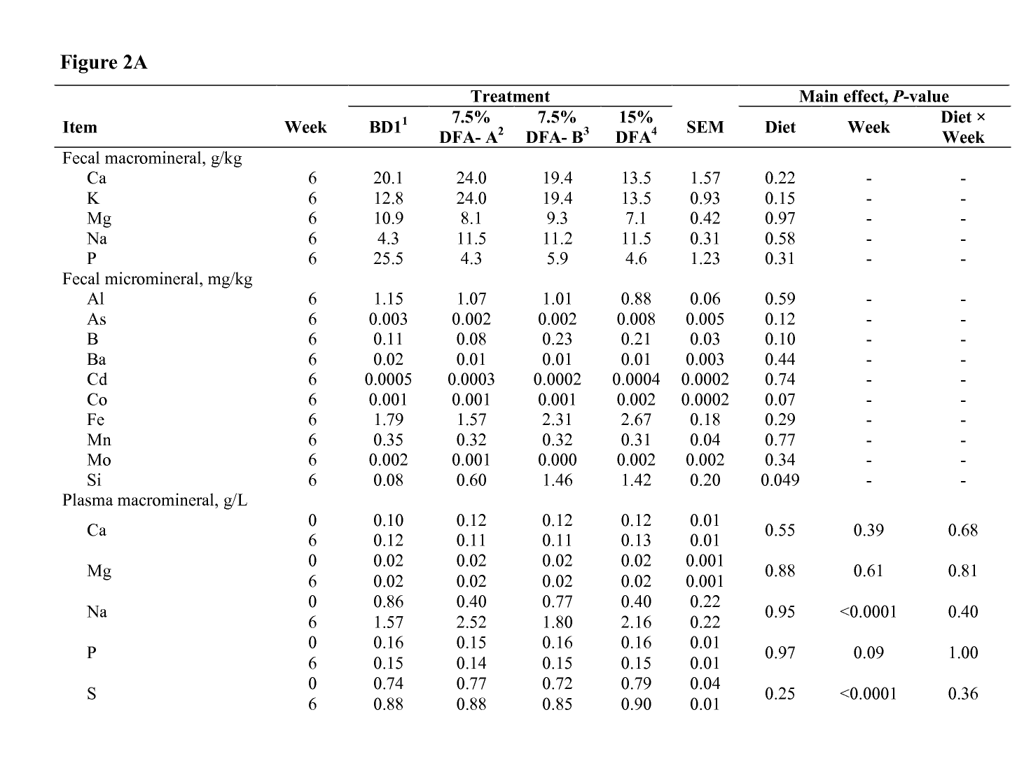

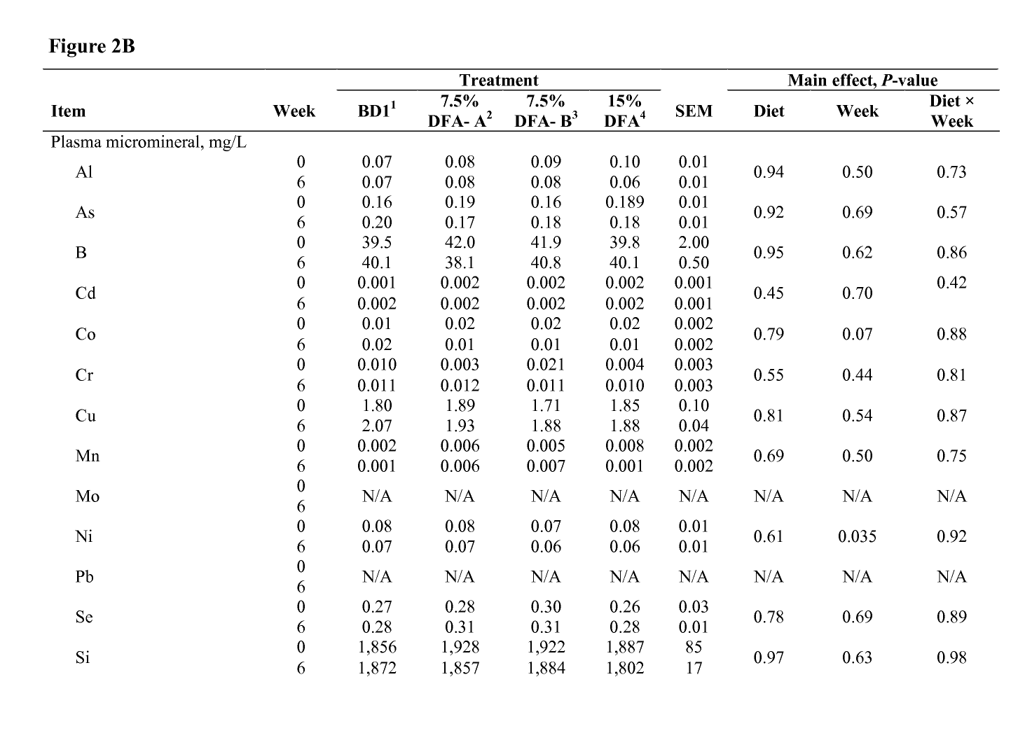

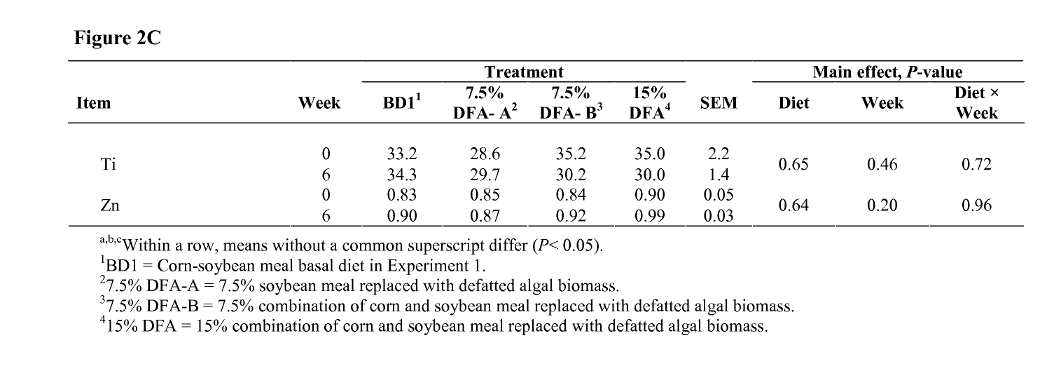

Inclusions of DFA elevated (P<0.0001 to 0.02) fecal mineral concentrations of S, Cr, Ni, Pb, Sr, and Ti, but decreased (P<0.05) fecal Cu, Se, and Zn (FIG. 1). No treatment differences were detected for all other fecal macro- and micro-minerals (FIGS. 2A-C). Pigs fed 15% DFA had 22%, 54%, and 61% greater (P=0.03) plasma Fe concentrations than those fed the BD1, 7.5% DFA-A, and 7.5% DFA-B, respectively. Plasma Sr concentration was elevated (P<0.0001) up to 10-fold in pigs fed the DFA-containing diets, as compared to pigs fed BD1. All other plasma macro- and micro-minerals showed no significant differences across all 4 treatment groups. There was an elevation trend in the fecal dry matter concentration with the increasing inclusion of DFA in the diets, where it was 12% greater (P=0.08) in pigs fed 15% DFA than those fed BD1.

Experiment 2

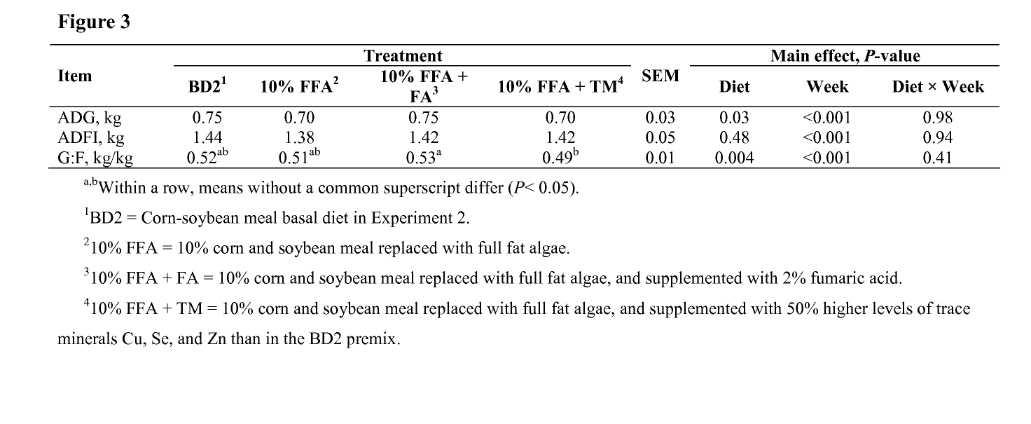

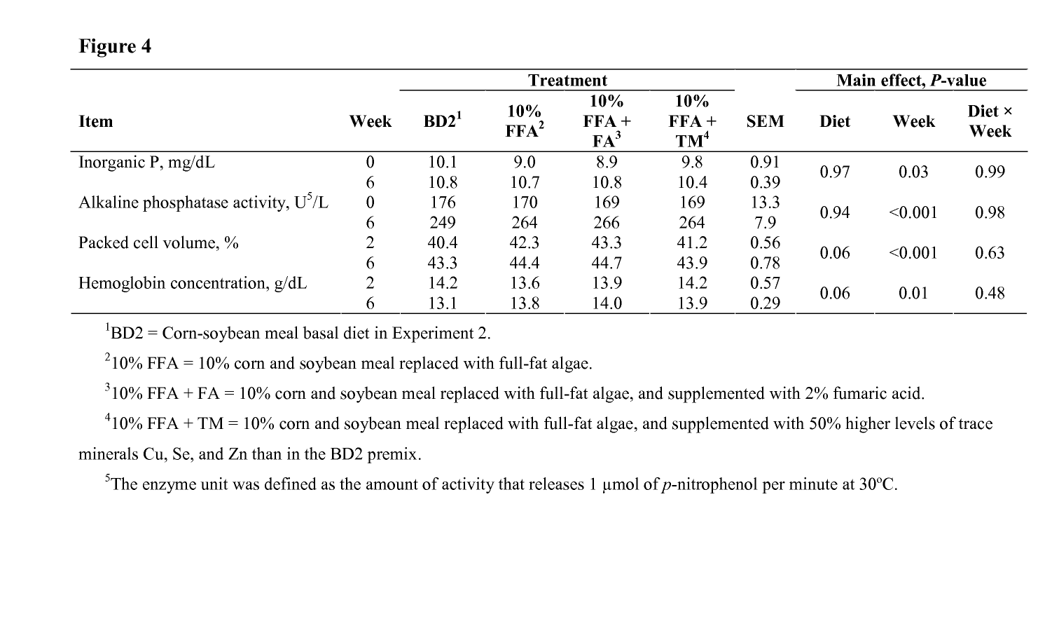

Compared with those fed BD2, overall ADG and G:F were 7% and 2% lower in pigs fed 10% FFA, 0.5% and 2% greater in pigs fed 10% FFA+FA, and 7% and 5% lower (P<0.03) in pigs fed 10% FFA+TM, respectively (FIG. 3). There was no difference in overall ADFI, plasma inorganic phosphorus, or plasma alkaline phosphatase activity among treatment groups (FIG. 4). However, there was a marginal increase in the overall packed cell volume (P=0.06) and hemoglobin concentration (P=0.06) in pigs fed the FFA-containing diets.

Discussion of Example 1

The main finding of Experiment 1 is that 7.5% DFA might be used to replace the same amounts of corn and SBM in diets of weanling pigs. Overall, this replacement did not affect growth performance or health status of pigs compared to those fed BD1. However, a replacement of either SBM alone with 7.5% DFA or a combination of corn and SBM with 15% DFA decreased ADG and G:F. The amounts of SBM removed from these DFA-containing diets might exceed the tolerance of pigs. Compared to BD1, 7.5% DFA-B only reduced the SBM level by 5.1%, whereas 7.5% DFA-A and 15% DFA reduced the level by 7.5% and 9%, respectively. Because DFA contained only 19% crude protein whereas SBM contained 47.5% crude protein, the higher levels of replacements of SBM might create protein and amino acid limitations. These limitations might account, in part, for the depressed growth performance.

Diatoms are the major phytoplankton characterized by silica in the outer membrane of their cell walls (Reimann, B. F., “Deposition of Silica Inside a Diatom Cell,” Exper. Cell Res. 34:605-608 (1964) and Popovskaya et al., “The Role of Endemic Diatom Algae in the Phytoplankton of Lake Baikal,” Hydrobiol. 568:87-94 (2006), which are hereby incorporated by reference in their entirety). Specifically, diatoms construct ornamented shells of amorphous silica that contain complex material in their frustule structure (Hecky et al., “The Amino Acid and Sugar Composition of Diatom Cell-Walls,” Marine Biol. 19:323-331 (1973) and Kroth, P., “Molecular Biology and the Biotechnological Potential of Diatoms,” Adv. Exper. Med. Biol. 616:23-33 (2007), which is hereby incorporated by reference in its entirety). Studies on diatoms show that in some species, total amino acids found in the cell wall are 1.2-fold greater than those found in the cell contents. As well, certain amino acids appear to be consistently enriched in the cell wall compared to the cell contents, such as serine, threonine, and glycine (Hecky et al., “The Amino Acid and Sugar Composition of Diatom Cell-Walls,” Marine Biol. 19:323-331 (1973), which is hereby incorporated by reference in its entirety).

Experiment 2 showed that supplementing 2% fumaric acid into the 10% FFA diet recovered the resultant losses in ADG and G:F. Proximate analyses of the DFA and FFA samples showed 45% and 40% ash content, respectively, and a nearly 20-fold increase in sodium, in contrast to either corn or SBM. As an alkali metal, sodium's heavy prominence in the microalgal biomass may largely skew the acid-base balance (Renner, T., “Acid-Base in Renal Failure: Influence of Diet on Acid-Base Balance,” Semin. Dial. 13:221-226 (2000), which is hereby incorporated by reference in its entirety) of the diet and thus the pigs. As such, the FFA-containing diet was supplemented with an acid to better neutralize the electrolyte balance and decrease the stomach pH of the pigs. Previous studies have shown that the supplementation of fumaric acid to the diets of young pigs improved their ADG and feed efficiency, as well as increased the apparent ileal digestibility of several amino acids and minerals (Kirchgessner et al., “Fumaric Acid as a Feed Additive in Pig Nutrition,” Pig News Info. 3:259 (1982); Falkowski et al., “Fumaric and Citric Acid as Feed Additives in Starter Pig Nutrition,” J. Anim. Sci. 58:935-938 (1984); Radecki et al., “Fumaric and Citric Acids as Feed Additives in Starter Pig Diets: Effect on Performance and Nutrient Balance,” J. Anim. Sci. 66:2598-2605 (1988); and Blank et al., “Effect of Fumaric Acid and Dietary Buffering Capacity on Ileal and Fecal Amino Acid Digestibilities in Early-Weaned Pigs,” J. Anim. Sci. 77:2974-2984 (1999), which are hereby incorporated by reference in their entirety). Because early-weaned pigs are somewhat incapable of secreting an adequate amount of HCl to maintain a low pH in their stomachs, the addition of fumaric acid may help the buffering capacity. In this way, digestive enzymes function under more optimal environmental conditions to better digest plant protein in the diet (Cranwell et al., “Gastric Secretion in Newly Born Pigs,” Res. Vet. Sci. 16:105-107 (1974); Kirchgessner et al., “Fumaric Acid as a Feed Additive in Pig Nutrition,” Pig News Info. 3:259 (1982); and Blank et al., “Effect of Fumaric Acid and Dietary Buffering Capacity on Ileal and Fecal Amino Acid Digestibilities in Early-Weaned Pigs,” J. Anim. Sci. 77:2974-2984 (1999), which are hereby incorporated by reference in their entirety). The positive growth performance response of pigs to the 10% FFA+2% fumaric acid indicated an effective strategy to improve the nutrient digestion of microalgal biomass.

Pigs fed the DFA-containing diets in Experiment 1 had lower fecal Cu, Se, and Zn concentrations than those fed BD1. Because there were no such differences in plasma concentrations of these elements, the fecal reduction might implicate a relative deficiency of these elements. Plausibly, this scenario could be arisen from an overestimation of bioavailable and digestible microalgal mineral concentrations used to formulate the diets. Although the ash content of DFA was high and its mineral profile was relatively well-balanced, the bioavailability and chemical forms of the minerals in DFA remain unknown. Alternatively, these decreases might reflect either an improved absorption of the minerals by pigs fed DFA, an increase in mineral requirements by the body, or a possible elevated excretion of urinal minerals. All these changes could lead to less fecal excretion of these minerals. However, supplementing 50% more Cu, Se, and Zn into the 10% FFA diet in Experiment 2 did not prevent the decreases of ADG or G:F. Seemingly, dietary supply or bioavailability of Cu, Se, and Zn was not a limiting factor in the algal replacement of corn and SBM. Alternatively, DFA and FFA did not share similar effects on Cu, Se, and Zn digestion or metabolism. Meanwhile, high ash contents in both DFA and FFA, along with the supplementation with extra trace minerals in both experiments produced no signs of toxicity in the animals. The bioavailability of minerals in microalgae may be limited owing to the rigid structure of the microalgal cell wall that comprises approximately 10% of the dry matter in most algae (Becker, E. W., Microalgae—Biotechnology and Microbiology, Cambridge: Cambridge University Press (1994), which is hereby incorporated by reference in its entirety). Little is known about the cell wall-specific mineral composition of diatom microalgae. Their minerals may be similarly presented within the cell wall, preventing effective digestion by simple-stomached animals.

A 22% higher plasma Fe concentration was observed in pigs fed 15% DFA than that of pigs fed BD1 in Experiment 1. Thus, the packed cell volume and blood hemoglobin concentration of pigs fed BD2 and the FFA-containing diets in Experiment 2 was compared. Consistently, both measures were affected by diet (P=0.06) and pigs fed the FFA-containing diets had numerically greater levels of these measures than those fed BD2. Likely, supplementing FFA improved dietary iron bioavailability for hemoglobin synthesis. With slightly greater levels of phosphorus analyzed in the DFA-containing diets than BD1 in Experiment 1, fecal total phosphorus and/or plasma inorganic phosphorus concentrations were determined in both experiments and no differences were found among all treatment groups.

These results clearly illustrate that a combination of corn and SBM may be replaced by either 7.5% DFA or 10% FFA+2% fumaric acid, without adverse effects on overall growth performance and plasma biochemical status in weanling pigs. The results on the fecal and plasma mineral profiles of the DFA-fed pigs are novel and may aid in understanding microalgae metabolism in animals. While feed application of algae was explored as early as in the 1950's (Grau et al., “Sewage-Grown Algae as a Feedstuff for Chicks,” Poult. Sci. 36:1046-1051 (1957); Gupta et al., “Studies on the Effect of Feeding Some Freshwater Fishes with Scenedesmusobliquus (Turpin) Kuetzing,” Hydrobiol. 28:42-48 (1966); Hintz et al., “Sewage-Grown Algae as a Protein Supplement for Swine,” Anim. Prod. 9:135-140 (1967); and He et al., “Supplementation of Algae to the Diet of Pigs: A New Possibility to Improve the Iodine Content in the Meat,” J. Anim. Physiol. 86:97-104 (2002), which are hereby incorporated by reference in their entirety), efforts in making algal biomass as a new generation of animal feed source are re-gaining worldwide support because of the rising global demands for alternative food and biofuel sources. A study by the United Soybean Board indicated that animal feeding in the U.S. alone used 27 million metric tons of SBM and 176 million metric tons of corn products and other ingredients in 2009-2010 per annum (United Soybean Board-Promar International (USB), Consumer and Food Safety Costs of Offshoring Animal Agriculture (2011), which is hereby incorporated by reference in its entirety). Although currently-available sources of microalgal biomass provide only 5,000 tons of dry matter per year (Spolaore et al., “Commercial Applications of Microalgae,” J. Biosci. Bioeng. 101:87-96 (2006), which is hereby incorporated by reference in its entirety), the innovative feed applications of the DFA, as shown in the present study, will help overcome the economical constraint of current biofuel production and generate substantial amount of microalgal biomass for animal feeding. Ultimately, a good portion of corn and SBM in animal diets can be spared for human consumption, which will promote sustainable animal agriculture and human food security.

### Example 2

### Defatted Diatom for Laying Hens: Dose-Dependent Effect of Defatted Diatom Biomass on Egg Production and Egg Quality of Laying Hens

Materials and Methods for Example 2

Animals, Dietary Treatments, and Management

A total of 100 ISA Babcock White Leghorn laying hens (47 weeks old, Gallus gallus domesticus) with an initial body weight of 1.57±0.20 kg, were randomly assigned to 4 dietary treatments. There were 5 replicates for each treatment and each replicate consisted of a row of 5 individually-caged hens in 60-cage units. The cages were equipped with nipple drinkers and trough feeders. The hen-house was provided with 16 hours of light per day and the hens were given free access to feed and water. The duration of the experiment was 8 weeks. The protocol for this research was approved by the Institutional Animal Care and Use Committee at Cornell University.

Defatted Staurosira sp. microalgal biomass used to prepare for the experimental diets was generated from the research on algal cultures for biofuel production (Cellana, Kailua-Kona, Hi.). Proximate and mineral analyses of the defatted algae were done by Dairy One, Inc. (Ithaca, N.Y.) and amino acid analyses were performed by Experiment Station Chemical Laboratories (University of Missouri, Columbia, Mo.). The composition of the defatted algae is presented in Table 10.

TABLE 10
Chemical Composition of Defatted Algal Biomass¹

| Item | Content |
|---|---|
| ME (Kcal/g) | 1.32 |
| Protein | 19.1 |
| Fat | 3.3 |
| Fiber | 14.7 |
| Ash | 44.9 |
| Moisture | 6.9 |
| Ca | 2.78 |
| P | 0.76 |
| Na | 3.94 |
| K | 1.66 |
| Cl | 6.34 |
| Arg | 0.93 |
| Lys | 0.83 |
| Met | 0.33 |
| Cys | 0.32 |
| Gly | 0.96 |
| Ser | 0.76 |
| His | 0.30 |
| Ile | 0.78 |
| Leu | 1.33 |
| Phe | 0.86 |
| Tyr | 0.57 |
| Thr | 0.88 |
| Trp | 0.18 |
| Val | 0.98 |

¹All items except ME are % of biomass (as fed basis).

The 4 experimental diets included a corn-soybean meal control diet (“CD”), the CD with 7.5% defatted algae and the 5 most limiting amino acids (Lys, Met, Ile, Thr, Tip) substituting for 7.5% soybean meal (7.5% algae-A), and the CD with 7.5% (7.5% algae-B) or 15% (15% algae) defatted algae substituting for mixture of soybean meal and corn (about 1:3.1). All diets were formulated to be isocaloric (2.80 Mcal of ME/kg, and iso-Ca, and iso-P by adjusting vegetable oil, dicalcium phosphate and limestone. Compositions and nutrient concentrations of the diets are presented in Table 11.

TABLE 11
Compositions and Nutrient Concentrations of the Diets

| | | 7.5% | 7.5% | 15% |
|---|---|---|---|---|
| Ingredients | Control | Algae-A | Algae-B | Algae |
| Corn (yellow) | 65.65 | 65.28 | 59.45 | 52.55 |
| Vegetable oils | 0.55 | 1.55 | 2.30 | 4.30 |
| Soybean meal | 22.50 | 15.00 | 20.50 | 18.50 |
| (48.5% crude protein) | | | | |
| Dicalcium phosphate | 1.50 | 1.40 | 1.35 | 1.20 |
| Limestone | 8.80 | 8.40 | 8.40 | 7.95 |
| Salt | 0.50 | 0.00 | 0.00 | 0.00 |
| Vitamin mix¹ | 0.25 | 0.25 | 0.25 | 0.25 |
| Mineral mix² | 0.15 | 0.15 | 0.15 | 0.15 |
| DL-Methionine | 0.10 | 0.10 | 0.10 | 0.10 |
| Defatted algae | 0.00 | 7.50 | 7.50 | 15.00 |
| Amino acids premix³ | 0.00 | 0.37 | 0.00 | 0.00 |
| Total | 100.00 | 100.00 | 100.00 | 100.00 |

Nutritional Composition (% except for ME)

| | | | | |
|---|---|---|---|---|
| ME (Kcal/g) | 2.80 | 2.80 | 2.80 | 2.80 |
| Protein | 16.49 | 14.26 | 16.43 | 16.30 |
| Fat | 3.27 | 4.73 | 5.31 | 7.58 |
| Fiber | 2.32 | 3.12 | 3.21 | 4.08 |
| Ca | 3.75 | 3.76 | 3.76 | 3.76 |
| P | 0.60 | 0.59 | 0.60 | 0.60 |
| Avail. P | 0.38 | 0.40 | 0.40 | 0.42 |
| Arg | 1.03 | 0.86 | 1.03 | 1.02 |
| Lys | 0.84 | 0.84 | 0.85 | 0.85 |
| Met | 0.36 | 0.36 | 0.38 | 0.39 |
| Cys | 0.28 | 0.23 | 0.26 | 0.23 |
| Gly | 0.68 | 0.61 | 0.70 | 0.73 |
| Ser | 0.80 | 0.68 | 0.80 | 0.79 |
| His | 0.44 | 0.36 | 0.42 | 0.40 |
| Ile | 0.67 | 0.67 | 0.68 | 0.69 |
| Leu | 1.50 | 1.33 | 1.48 | 1.46 |
| Phe | 0.78 | 0.67 | 0.78 | 0.78 |
| Tyr | 0.64 | 0.55 | 0.64 | 0.64 |
| Thr | 0.61 | 0.61 | 0.63 | 0.65 |
| Trp | 0.21 | 0.21 | 0.21 | 0.22 |
| Val | 0.76 | 0.68 | 0.78 | 0.79 |

¹Provided (in mg/kg of diet): CuSO₄•5H₂O, 31.42; KI, 0.046; FeSO₄•7H₂O, 224.0; MnSO₄•H₂O, 61.54; Na₂SeO₃, 0.13; ZnO, 43.56; Na₂MoO₄•2H₂O, 1.26.
²Provided (in IU/kg of diet): vitamin A, 6500; vitamin D₃, 3500; vitamin E, 25 and (in mg/kg of diet): riboflavin, 25; d-calcium pantothenate, 25; nicotinic acid, 150; cyanocobalamin, 0.011; choline chloride, 1250; biotin, 1.0; folic acid, 2.5; thiamine hydrochloride, 7.0; pyridoxine hydrochloride, 25.0; menadione sodium bisulfite, 5.0; and ethoxyquin, 66.
³Provided 1.8 g L-Lys•HCL, 0.1 g DL-Met, 0.9 g L-Ile, 0.6 g L-Thr, 0.3 g L-Trp per kilogram of diet.

Sample Collection and Procedures

Body weights of the laying hens were recorded at the start and the end of the experiment. Eggs were collected daily and egg production was calculated on a hen-day basis. Feed intake was recorded weekly by replicate. Eggs produced on the last 3 days of the 4^{th} week and 8^{th} week were individually weighed and analyzed for their interior and exterior quality. Eggs were examined for shell quality by measuring egg shell thickness, breaking strength, and egg specific gravity. Shell thickness, without shell membrane, was measured by micrometer with a mean value of measurements at 3 locations on the eggs (near the equator). The strength of egg shells was determined as the compression pressure necessary to crack the shell when the egg was placed horizontally between the plates of an Instron model 5969 (Instron, Norwood, Mass.). The specific gravity of eggs was determined by the buoyancy of eggs in salt solutions of varying density. Egg components, including albumen, yolk, and shell were weighed separately. Haugh units, a measure of the height of the albumen of eggs broken out on a flat surface, were determined by the use of a micrometer (U.S. Department of Agriculture, Consumer and Marketing Service), “Egg Grading Manual. Agriculture Handbook,” Washington, D.C., NO 75 (1969), which is hereby incorporated by reference in its entirety). Yolk color, measured as L*-, a*-, and b*-values, was determined with a Macbeth Color Eye (Macbeth Division of Kollmorgen Instruments Corp., Newburgh, N.Y.). The L* value represents lightness (negative towards black, positive towards white), the a* value red-greenness (negative towards green, positive towards red) and the b* value the blue-yellow color scale (negative towards blue, positive towards yellow).

Uric acid, alanine transaminase (“ALT”), cholesterol, and glucose in plasma were determined with Uric acid liquid stable reagent (Infinity TM, Fisher Diagnostics, Fisher Scientific Company, LLC, Middletown, N.Y.), ALT Liquid stable reagent (Thermo Electron Corporation, Pittsburgh, Pa.), Cholesterol reagent (Wako Pure Chemical Industries, Ltd, Osaka, Japan), and Glucose Assay Kit (Sigma, St. Louis, Mo.), respectively. Alkaline phosphatase (“AKP”) was determined by the method of Bowers and McComb (Bowers et al., “A Continuous Spectrophotometric Method for Measuring the Activity of Serum Alkaline Phosphatase,” Clin. Chem. 12:70-89 (1966), which is hereby incorporated by reference in its entirety). Yolk cholesterol was extracted by the method of Folch et al. (Folch et al., “A Simple Method for the Isolation and Purification of Total Lipides from Animal Tissues,” J. Biol. Chem. 226:497-509 (1957), which is hereby incorporated by reference in its entirety) and measured by the HPLC method of Beyer and Jensen (Beyer et al., “Overestimation of the Cholesterol Content of Eggs,” J. Agric. Food Chem. 37:917-920 (1989), which is hereby incorporated by reference in its entirety). Lipids were extracted from 1.00 g samples of yolk using a modification of the method of Bligh and Dyer (Bligh et al., “A Rapid Method of Total Lipid Extraction and Purification,” Can. J. Biochem. Physiol. 37(8):911-917 (1959), which is hereby incorporated by reference in its entirety). Fatty acid methyl esters (“FAME”) were prepared by trans-esterification (Metcalf et al., “Rapid Preparation of Fatty Acid Esters from Lipids for Gas Chromatographic Analysis,” Anal. Chem. 38(3):514-515 (1966), which is hereby incorporated by reference in its entirety), dissolved in hexane, and stored under refrigeration in amber chromatography vials. One microliter samples were manually injected, using a 40:1 split in helium carrier gas into a 3610C Series GLC (SRI Instruments, Torrance, Calif.) equipped with a 60 meter×0.32 mm I. D. BPX-70 column with 0.25 μm film (Phenomenex, Torrance, Calif.), flame ionization detector, and Peak Simple software. The injector and detector temperatures were 270° C. and 350° C., respectively. The temperature program was 50° C. (initial temperature) held 0.1 min and then ramped 50° C./min to 170° C. and held 7 minutes. The temperature was then ramped 4° C./min to 200° C. without hold and then ramped 50° C./min to 255° C. and held 10 minutes before returning to 50° C. for injection of the subsequent sample. A 20 component marine oil FAME mixture (Restek Corporation, Bellefonte, Pa.) was used as an external standard. Using peak area responses of the external standard, the individual peak areas in each sample chromatogram were converted to gram equivalents and expressed as the % of total FAME.

Statistical Analysis

The experimental data were presented as mean±standard error using SPSS17.0 (Prentice Hall, Inc., Upper Saddle River, N.J.) for one-way analysis of variance, combined with Duncan's method for multiple comparisons. The significance level for differences was P<0.05.

Results for Example 2

Body Weight, Feed Intake, and Egg Production Rate

There were no significant differences in body weight among treatment groups at the beginning or the end of the experiment, despite an average loss of 20 to 70 g/hen during the experiment across all groups (Table 12). In the first 4 weeks the inclusion of defatted algae tended to decrease feed intake and egg production rate. In the second 4 weeks, hens fed 15% defatted algae had lower (P<0.05) feed intake and egg production rate than those of the control group. During the whole 8-week period, hens fed 15%, but not 7.5% defatted algae had a lower (P<0.05) egg production rate (−12%) and daily feed intake (−9 g/hen/d) than those fed the control diet (Table 12). There were no significant differences in plasma AKP, ALT, cholesterol, or glucose among groups at the end of the study (the 8^{th} week). However, hens fed 15% DFA had lower (P, 0.05) plasma uric acid level than that of control group.

TABLE 12
Effect of Defatted Algae on Body Weight,
Feed Intake, and Egg Production Rate

| | | | |
|---|---|---|---|
| | 7.5% | 7.5% | 15% |
| Control | Algae-A | Algae-B | Algae |

Body weight (kg)

| | | | | |
|---|---|---|---|---|
| Wk 0 | 1.53 ± 0.16 | 1.60 ± 0.19 | 1.58 ± 0.18 | 1.58 ± 0.20 |
| Wk 8 | 1.47 ± 0.08 | 1.58 ± 0.08 | 1.53 ± 0.11 | 1.51 ± 0.10 |

Daily feed intake (g/hen)

| | | | | |
|---|---|---|---|---|
| Wk 1-4 | 100.1 ± 4.9 | 96.9 ± 3.9 | 95.9 ± 6.7 | 92.6 ± 11.2 |
| Wk 5-8 | 96.0 ± 8.3^{1a} | 96.0 ± 3.6^{a} | 95.0 ± 6.9^{a} | 85.5 ± 4.5^{b} |
| Wk 1-8 | 98.1 ± 6.0^{a} | 96.4 ± 3.4^{ab} | 95.4 ± 5.9^{ab} | 89.1 ± 5.9^{b} |

Egg production rate (%)²

| | | | | |
|---|---|---|---|---|
| Wk 1-4 | 86.7 ± 3.4 | 83.7 ± 6.3 | 84.7 ± 6.6 | 75.1 ± 16.4 |
| Wk 5-8 | 81.3 ± 11.2^{a} | 85.6 ± 5.0^{a} | 83.9 ± 8.7^{a} | 69.0 ± 6.6^{b} |
| Week 1-8 | 84.0 ± 7.1^{a} | 84.6 ± 4.7^{a} | 84.3 ± 7.1^{a} | 72.1 ± 10.1^{b} |

¹Means in the same row with different letters are different (P < 0.05).
²Egg production = 100 [number of eggs laid ÷ (number of hens × number of days)].

Egg Quality and Yolk Color

At the end of 4^{th} and 8^{th} weeks, there were no significant differences between the control and the DFA diets in egg quality indexes (Table 13), including egg weight, Haugh unit, yolk weight, shell weight, albumen weight, and shell thickness. However, the 15% DFA diet had higher (P<0.05) egg albumen weight and height than the 7.5% Algae-A and -B diet, respectively, at the 8^{th} week. Additionally, the DFA diets produced no difference from the control diet in egg specific gravity or egg shell breaking strength at the end of study (the 8^{th} week).

TABLE 13
Effect of Defatted Algae on Egg Quality

| | | | |
|---|---|---|---|
| | 7.5% | 7.5% | 15% |
| Control | Algae-A | Algae-B | Algae |

At the end of the 4^{th} week

| | | | | |
|---|---|---|---|---|
| Egg weight (g) | 60.00 ± 3.93 | 59.49 ± 5.25 | 59.28 ± 3.79 | 60.69 ± 3.61 |
| Haugh unit | 86.6 ± 4.0 | 84.6 ± 6.4 | 85.6 ± 7.1 | 88.6 ± 5.7 |
| Yolk weight (g) | 15.79 ± 1.42 | 16.35 ± 1.42 | 15.77 ± 1.22 | 15.92 ± 0.82 |
| Egg shell weight (g) | 5.29 ± 0.57 | 5.70 ± 2.11 | 5.37 ± 0.39 | 5.60 ± 0.54 |
| Albumin weight (g) | 38.81 ± 2.96 | 38.21 ± 3.89 | 38.13 ± 2.83 | 39.00 ± 3.24 |
| Egg shell thickness (mm) | 0.34 ± 0.03 | 0.34 ± 0.03 | 0.36 ± 0.03 | 0.36 ± 0.03 |

At the end of the 8^{th} week

| | | | | |
|---|---|---|---|---|
| Egg weight (g) | 59.99 ± 2.51 | 58.09 ± 1.49 | 59.25 ± 1.75 | 60.05 ± 1.26 |
| Haugh unit | 82.6 ± 3.1 | 82.0 ± 3.3 | 81.6 ± 2.0 | 84.6 ± 1.4 |
| Yolk weight (g) | 16.11 ± 0.71 | 16.25 ± 0.55 | 16.08 ± 0.48 | 15.90 ± 0.32 |
| Egg shell weight (g) | 5.47 ± 0.26 | 5.28 ± 0.25 | 5.38 ± 0.25 | 5.49 ± 0.35 |
| Albumin weight (g) | 38.44 ± 2.17^{1ab} | 36.78 ± 1.40^{a} | 37.87 ± 1.45^{ab} | 39.24 ± 1.15^{b} |
| Albumn height (mm) | 6.92 ± 0.43^{ab} | 6.73 ± 0.47^{ab} | 6.71 ± 0.24^{a} | 7.23 ± 0.23^{b} |
| Egg shell thickness (mm) | 0.35 ± 0.02 | 0.35 ± 0.01 | 0.34 ± 0.03 | 0.35 ± 0.02 |
| Egg specific gravity | 1.082 ± 0.004 | 1.080 ± 0.001 | 1.080 ± 0.005 | 1.080 ± 0.002 |
| Egg shell strength (N) | 34.4 ± 2.2 | 34.4 ± 2.0 | 32.6 ± 4.6 | 35. 3 ± 3.6 |

¹Means in the same row with different letters are different (P < 0.05).

Including algae in the diets affected (P<0.05) yolk color in a dose-dependent fashion (Table 14). The L* (lightness) value and +b* (yellowness) value of yolk decreased (P<0.05) with the increasing algae content of the diet. At the 8^{th} week, but not the 4^{th} week, the values for lightness and yellowness were lower (P<0.05) for 7.5% algae-B than for 7.5% algae-A. The +a* (redness) values of yolk were elevated (P<0.05) by all three DFA diets at the end of the 4^{th} week, but by only the two 7.5% DFA diets at the 8^{th} week.

TABLE 14
Effect of Defatted Algae on Yolk Color

| | | | |
|---|---|---|---|
| | 7.5% | 7.5% | 15% |
| Control | Algae-A | Algae-B | Algae |

At the end of the 4^{th} week

| | | | | |
|---|---|---|---|---|
| L*(lightness) | 54.806 ± 0.541^{1a} | 52.502 ± 0.602^{b} | 51.943 ± 0.650^{b} | 50.328 ± 0.878^{c} |
| +a*(redness) | 11.576 ± 0.209^{a} | 13.438 ± 0.305^{b} | 13.316 ± 1.015^{b} | 12.939 ± 0.329^{b} |
| +b*(yellowness) | 35.660 ± 0.314^{a} | 34.522 ± 0.320^{b} | 34.013 ± 0.383^{b} | 33.038 ± 0.558^{c} |

At the end of the 8^{th} week

| | | | | |
|---|---|---|---|---|
| L*(lightness) | 54.758 ± 0.430^{a} | 52.718 ± 0.393^{b} | 51.690 ± 1.014^{c} | 50.732 ± 0.762^{d} |
| +a*(redness) | 11.084 ± 0.317^{a} | 12.573 ± 0.288^{b} | 12.518 ± 0.889^{b} | 11.356 ± 0.607^{a} |
| +b*(yellowness) | 35.479 ± 0.238^{a} | 34.499 ± 0.149^{b} | 33.697 ± 0.561^{c} | 32.856 ± 0.239^{d} |

¹Means in the same row with different letters are different (P < 0.05).

Plasma Biochemical Indices

Plasma biochemical indices of laying hens after 8 weeks of experiment are shown in Table 15. There were no significant differences in plasma AKP, ALT, cholesterol, or glucose among groups. Hens of the 15% algae group had lower (P<0.05) uric acid level than that of control group.

TABLE 15
Effect of Defatted Algae on Plasma Bio-
chemical Indices of Hens at the 8^{th} Week

| | | 7.5% | 7.5% | 15% |
|---|---|---|---|---|
| | Control | Algae-A | Algae-B | Algae |
| AKP | 111.5 ± 29.0 | 146.4 ± 34.1 | 155.4 ± 44.2 | 133.8 ± 31.1 |
| (U/ml) | | | | |
| ALT | 24.1 ± 6.5 | 24.3 ± 9.1 | 33.5 ± 15.8 | 29.5 ± 7.4 |
| (U/L) | | | | |
| Choles- | 117.5 ± 8.5 | 117.4 ± 12.3 | 112.6 ± 22.5 | 106.0 ± 15.6 |
| terol | | | | |
| (mg/dL) | | | | |
| Glucose | 204.9 ± 4.6 | 210.7 ± 13.4 | 209.1 ± 9.4 | 198.6 ± 7.0 |
| (mg/dL) | | | | |
| Uric acid | 10.7 ± 1.9^{a} | 10.2 ± 2.7^{ab} | 8.6 ± 2.4^{ab} | 7.6 ± 1.3^{b} |
| (mg/dL) | | | | |

¹Means in the same row with different letters are different (P < 0.05).

Egg Yolk Lipids

The composition of egg yolk lipids are shown in Table 16. The 4 treatment groups had similar egg yolk cholesterol contents, but displayed different egg yolk fatty acid profiles. The proportion of palmitoleic acid (C16:1) decreased (P<0.05) with increased dietary levels of algae biomass, and the proportion of oleic acid (C18:1) decreased (P<0.05) with the 15% algae inclusion. The proportion of linoleic acid (C18:2) was in the order of 15% algae>7.5% algae-B>the control (P<0.05). Linolenic (C18:3) was higher (P<0.05) in the 7.5% algae-A group than in the control group.

TABLE 16
Effect of Defatted Algae on Yolk Cholesterol
and Fatty Acid Composition at the 8^{th} Week

| | | | |
|---|---|---|---|
| | 7.5% | 7.5% | 15% |
| Control | Algae-A | Algae-B | Algae |

Cholesterol (mg/g tissue)

| | | | |
|---|---|---|---|
| 14.20 ± 0.66 | 14.06 ± 0.34 | 13.50 ± 0.66 | 13.69 ± 0.71 |

Fatty acid profile (weight percent of fatty acids)

| | | | | |
|---|---|---|---|---|
| C14:0 | 0.49 ± 0.08 | 0.40 ± 0.10 | 0.41 ± 0.10 | 0.46 ± 0.08 |
| C14:1 | 0.13 ± 0.03 | 0.11 ± 0.01 | 0.13 ± 0.10 | 0.08 ± 0.05 |
| C16:0 | 31.88 ± 2.23 | 29.40 ± 2.62 | 29.48 ± 2.44 | 29.21 ± 2.01 |
| C16:1 | 4.52 ± 0.38^{1a} | 3.82 ± 0.44^{b} | 3.90 ± 0.48^{b} | 3.11 ± 0.30^{c} |
| C18:0 | 8.23 ± 2.1 | 8.13 ± 1.08 | 9.06 ± 3.07 | 7.61 ± 1.15 |
| C18:1 | 36.52 ± 2.40^{a} | 36.46 ± 1.29^{a} | 34.57 ± 2.24^{ab} | 33.12 ± 1.99^{b} |
| C18:2 | 13.70 ± 1.43^{c} | 15.90 ± 2.97^{bc} | 17.28 ± 1.70^{b} | 22.32 ± 0.49^{a} |
| C18:3 | 0.34 ± 0.13^{b} | 1.01 ± 0.46^{a} | 0.67 ± 0.42^{ab} | 0.34 ± 0.09^{b} |
| C20:0 | 1.59 ± 1.22 | 2.87 ± 0.69 | 1.23 ± 1.10 | 1.37 ± 1.42 |
| C20:1 | 0.70 ± 0.79 | 0.79 ± 0.63 | 0.70 ± 0.60 | 0.44 ± 0.41 |
| C20:4 | 1.32 ± 0.23 | 1.42 ± 0.20 | 1.57 ± 0.22 | 1.49 ± 0.12 |
| C22:6 | 0.59 ± 0.15 | 0.81 ± 0.35 | 0.57 ± 0.19 | 0.59 ± 0.12 |

¹Means in the same row with different letters are different (P < 0.05).

Discussion for Example 2

In the present study, 7.5% defatted algae biomass replaced 7.5% soybean meal, or a combination of corn and soybean meal in diets without any adverse effects on hen body weights, feed consumption, and egg production. The inclusion of 15% defatted algae biomass reduced feed intake and egg production. During the experiment, hens in the 15% defatted algae biomass group were observed to drink more water and have a larger volume of feces. Many marine algae have high ash contents, which could be more problematic in the biomass remaining after lipid extraction. The defatted diatom in this study contained 44.9% ash and 10.28% sodium chloride. These two properties likely account for the bulky wet droppings and may have contributed to lower feed intake in the 15% algae group.

Past studies on inclusions of algae in diets for poultry indicate that algae can be used safely at dietary levels of 5% to 10% (Combs, G. F., “Algae (Chlorella) as a Source of Nutrients for the Chick,” Science. 116:453-454 (1952); Grau et al., “Sewage-Grown Algae as a Feedstuff for Chicks,” Poult. Sci. 36:1046-1051 (1957); Yoshida et al., “Nutritive Value of New Type of Chlorella for Poultry Feed,” Jpn. Poult. Sci. 19:56-58 (1982); Lipstein et al., “The Nutritional Value of Sewage-Grown Samples of Chlorella and Micractinium in Broiler Diets,” Poult. Sci. 62:1254-1260 (1983); Ross et al., “The Nutritional Value of Dehydrated, Blue-Green Algae (Spirulina platensis) for Poultry,” Poult. Sci. 69:794-800 (1990); Venkataraman et al., “Replacement Value of Blue-Green Alga (Spirulina platensis) for Fishmeal and a Vitamin-Mineral Premix for Broiler Chicks,” Br. Poult. Sci. 35:373-381 (1994); Halle et al., “Effect of Microalgae Chlorella vulgaris on Laying Hen Performance,” Arch. Zootech. 12(2):5-13 (2009), which are hereby incorporated by reference in their entirety).

Egg weights were not affected by defatted algae in the present experiment. These results are consistent with other reports on the use of 5% to 10% algae in diets of chickens (Lipstein et al., “The Nutritional Value of Algae for Poultry. Dried Chlorella in Layer Diets,” Br. Poult. Sci. 21:23-27 (1980); Lipstein et al., “The Nutritional Value of Sewage-Grown, Alum Flocculated Micractinium Algae in Broiler and Layer Diets,” Poult. Sci. 60:2628-2638 (1981), which are hereby incorporated by reference in their entirety) and quail (Ross et al., “The Nutritional Value of Dehydrated, Blue-Green Algae (Spirulina platensis) for Poultry,” Poult. Sci. 69:794-800 (1990); Halle et al., “Effect of Microalgae Chlorella vulgaris on Laying Hen Performance,” Arch. Zootech. 12(2):5-13 (2009), which are hereby incorporated by reference in their entirety) fed up to 7.5 g Chlorella per kilogram of feed. In their experiments, yolk weight of the hens given algae increased by about 11%, albumen weight was decreased by 8%. No negative effects of defatted algae on of egg shell weight, egg shell thickness, egg breaking strength, or firmness of the albumen (Haugh Units) were observed in the present experiment. The results are consistent with other reports involving chickens (Lipstein et al., “The Nutritional Value of Sewage-Grown Samples of Chlorella and Micractinium in Broiler Diets,” Poult. Sci. 62:1254-1260 (1983); Halle et al., “Effect of Microalgae Chlorella vulgaris on Laying Hen Performance,” Arch. Zootech. 12(2):5-13 (2009); Lipstein et al., “The Nutritional Value of Sewage-Grown, Alum Flocculated Micractinium Algae in Broiler and Layer Diets,” Poult. Sci. 60:2628-2638 (1981); Sauveur et al., “Protéines Alimentaires et qualité de l'oeuf. I. Effet de quelques protéines sur la qualité interne de l'oeuf et les propriétes fonctionnelles,” Ann. Zootech. 28(3):271-295 (1979), which are hereby incorporated by reference in their entirety) and Japanese quail Ross et al., “The Nutritional Value of Dehydrated, Blue-Green Algae (Spirulina platensis) for Poultry,” Poult. Sci. 69:794-800 (1990), which is hereby incorporated by reference in its entirety) in which one or more of these traits were reported.

Carotenoids are responsible for a wide variety of colors they provide in nature. Although algae powder appears as a bluish-green color, in fact it contains high levels of carotenoids such as β-carotene, lutein, and zeaxanthin (Miki et al., “Carotenoid Composition of Spirulina Maxima,” Bull. Jpn. Sco. Sci. Fish. 52(7):1225-1227 (1986), which is hereby incorporated by reference in its entirety). The red carotenoid astaxanthin is present in some algae (Spolaore et al., “Commercial Applications of Microalgae,” J. Biosci. Bioeng. 101(2):87-96 (2006), which is hereby incorporated by reference in its entirety). Because poultry and other animals cannot synthesize carotenoids de novo, the color of broiler skin and leg and egg yolk can be enhanced by including algae in feeds (Becker W. In: Handbook of Microalgal Culture: Biotechnology and Applied Phycology. Richmond, A. (Ed), Microalgae in Human and Animal Nutrition, Oxford, Blackwell Science, pp. 312-351 (2004), which is hereby incorporated by reference in its entirety). When 10% and 20% algae (Nannochloropsis oculata) was included in the diet, the yolk redness (+a* value) increased from −4.7 to 12.1, and 16.1, total carotenoids in yolk increased from 9.1 to 35.5, and 42.1 mg/kg, but yolk yellowness was not substantially altered by the algae (Fredriksson et al., “Fatty Acid and Carotenoid Composition of Egg Yolk as an Effect of Microalgae Addition to Feed Formula for Laying Hens,” Food Chem. 99:530-537 (2006), which is hereby incorporated by reference in its entirety). In the present study, 7.5% defatted algae in diet increased the +a (redness) value and decreased+b (yellowness) value of yolk. The higher inclusion level (15%) further decreased yolk yellowness. Similar increases in redness in response to marine algae have been reported by Herber-McNeill and Van Elswyk (Herber-McNeill et al., “Dietary Marine Algae Maintains Egg Consumer Acceptability While Enhancing Yolk Color,” Poult. Sci. 77:493-496 (1998), which is hereby incorporated by reference in its entirety). These results indicate the presence of the red pigment astaxanthin in the defatted diatom.

The present study showed no yolk cholesterol alteration or enrichment of eggs with omega-3 fatty acids by the defatted algae products. Marine algae have been investigated as a source of lipids for enrichment of eggs and poultry with omega-3 fatty acids. Barclay et al. supplemented the diet of laying hens with 1.2% dried product from the culture of a species of Schizochytrium (Barclay et al. In: The return of ω3 Fatty Acids into the Food Supply. I. Land-based Animal Food Products and their Health Effects, Simopoulos, A. P. (Ed), “Production of Docosahexaenoic Acid from Microalgae and Its Benefits for Use in Animal Feeds,” World Rev. Nutr. Diet. Basil, Karger 83:61-76 (1998), which is hereby incorporated by reference in its entirety), and found that the daily intake of 165 mg of docosahexaenoic acid (“DHA”) resulted in the deposition of 150 mg of DHA per egg. In a subsequent study, Abril and Barclay demonstrated that providing 300 or 600 mg/hen/day of DHA as the dried algal product increased total omega-3 fatty acids from 40 mg/egg in the control group to 172 mg/egg and 243 mg/egg, respectively, after 39 days of experiment (Abril et al., In: The Return of ω3 Fatty Acids into the Food Supply. I. Land-Based Animal Food Products and Their Health Effects, Simopoulos AP (ed), “Production of Docosahexaenoic Acid-Enriched Poultry Eggs and Meat Using an Algae-based Feed Ingredient,” World Rev. Nutr. Diet. Basal, Karger 83:77-88 (1998), which is hereby incorporated by reference in its entirety). Other investigators have also reported omega-3 fatty acid enrichments of eggs with algal products (Nitsan et al., “Enrichment of Poultry Products with ω3 Fatty Acids by Dietary Supplementation with the Alga Nannochloropsis and Mantur Oil,” J. Agric. Food Chem. 47:5127-5132 (1999); Herber-McNeill et al., “Dietary Marine Algae Maintains Egg Consumer Acceptability While Enhancing Yolk Color,” Poult. Sci. 77:493-496 (1998), which are hereby incorporated by reference in their entirety). The failure to detect eicosapentaenoic acid (“EPA”) or increases in other omega-3 fatty acids in egg yolk in the present experiment may be attributed to the low level of fat in the defatted algae and the addition of substantial levels of dietary corn oil to the diets to maintain energy equivalency.

Several genera of algae have been analyzed for protein quality based on non-protein urea and biological value (Becker W. In: Handbook of Microalgal Culture: Biotechnology and Applied Phycology, Richmond, A. (Ed), “Microalgae in Human and Animal Nutrition,” Oxford, Blackwell Science, pp. 312-351 (2004); Becker, E. W., “Micro-Algae as a Source of Protein,” Biotech. Adv. 25:207-210 (2007), which are hereby incorporated by reference in their entirety). Although the results indicate that protein quality varies among algae, many species have relatively high protein content and excellent protein quality by these measures. In the present experiment, the defatted diatom was added to the diet in replacement of and equivalent weight of soybean meal or a mixture of soybean and corn. The defatted diatom contains 19% crude protein in contrast to 48% crude protein in soybean meal. Substitution of one-third of the soybean meal with defatted diatom necessarily means that the resulting diet had less protein than the control. Several amino acids were added to the 7.5% Algae-A diet in an attempt to meet amino acid requirements. This diet supported production responses that were similar to those of the control diet. Only uric acid, among the serum biomarkers, was affected by the algae treatments. The decrease in uric acid concentration in hens given the diet containing 15% algae may be a reflection of the 15% lower daily feed consumption during the last 4 weeks of experiment. Uric acid is the primary end product of amino acid catabolism in birds. The lower intake of dietary protein might have resulted in a reduction in the amount of protein that was subject to catabolism. Alternatively, the low uric acid excretion might reflect a low digestibility and/or a better utilization of protein in the algae as compared to that of protein in the other dietary ingredients.

### Example 3

### Potential and Limitation of a New Defatted Diatom Microalgal Biomass in Replacing Soybean Meal and Corn in Diets for Broiler Chickens

Materials and Methods for Example 3

Animals, Diets, and Management

The protocols of all experiments were approved by the Institutional Animal Care and Use Committee of Cornell University. Hatchling Ross broiler chicks were obtained from a commercial hatchery and housed in a temperature-controlled room at the Cornell University Poultry Research Farm. The day-old broiler chicks were housed in thermostatically-controlled cage batteries for 3 weeks and were transferred to grower cages at room temperature from 3 to 6 weeks. Chicks had free access to feed and water and were provided with a lighting schedule of 22 hours light, 2 hours dark daily. Body weights were recorded at the beginning of experiments, and were recorded weekly thereafter, along with feed intake. The DFA of Staurosira sp. (Table 17) (Cellana, Kailua-Kona, Hi.) was included at levels of 7.5% or 10% of the diets in partial substitution for SBM or a combination of SBM and ground corn. Crystalline amino acids, minerals and vitamins were added to satisfy nutrient requirements. Starter (0-3 weeks) and grower diets (4-6 weeks) were designed to meet the requirements for growth for each age group (National Research Council, Nutrient Requirements of Poultry, 9th revised Edition, National Academy Press, Washington D.C. (1994), which is hereby incorporated by reference in its entirety). Diets containing the same amount of DFA used in different experiments were similar in the major ingredients but differed in amino acid concentrations. As examples, the control starter and grower diets and the diets containing alga substituting for SBM in Experiment 1 are shown in Table 18. Dietary treatments and the duration of experiments differed, but the animal housing and feeding protocols were similar among experiments.

TABLE 17
Chemical Composition of the Defatted
Diatom Microalgal Biomass (DFA)¹

| Item | Content |
|---|---|
| ME(Kcal/g) | 1.32 |
| Protein | 19.1 |
| Fat | 3.3 |
| ND Fiber² | 14.0 |
| AD Fiber² | 0.7 |
| Ash | 44.9 |
| Moisture | 6.9 |
| Calcium (Ca) | 2.78 |
| Phosphorus (P) | 0.76 |
| Sodium (Na) | 3.94 |
| Potassium (K) | 1.66 |
| Magnesium (Mg) | 0.79 |
| Chloride Cl) | 6.34 |
| Iron (Fe) | 1820 |
| Copper (Cu) | 4 |
| Manganese (Mn) | 101 |
| Zinc (Zn) | 25 |
| Molybdenum (Mo) | 2.2 |
| Selenium (Se) | <0.01 |
| Taurine | 0.08 |
| Lanthionine | 0.00 |
| Arginine | 0.93 |
| Lysine | 0.83 |
| Methionine | 0.33 |
| Cysteine | 0.32 |
| Glycine | 0.96 |
| Serine | 0.76 |
| Histidine | 0.3 |
| Isoleucine | 0.78 |
| Leucine | 1.33 |
| Phenylalanine | 0.86 |
| Tyrosine | 0.57 |
| Threonine | 0.88 |
| Tryptophan | 0.18 |
| Valine | 0.98 |
| Alanine | 1.09 |
| Aspartic acid | 1.88 |
| Glutamic acid | 1.81 |
| Proline | 0.65 |
| Hydroxyproline | 0.04 |
| Hydroxylysine | 0.3 |
| Ornithine | 0.03 |

¹All values are on an “as is” basis. Values other than ME and the trace elements (Fe, Cu, Mn, Zn, Mo, and Se) are expressed as % of biomass. These six trace elements are expressed in mg/kg of biomass.
²ND, neutral detergent; AD, acid detergent.

TABLE 18
Typical Composition of Control and 7.5% Algae-containing Diets

| | Starter diets | | Grower diets | |
|---|---|---|---|---|
| | (0-3 week) | | (4-6 weeks) | |
| | | 7.5% | | 7.5% |
| Ingredients | Control | Algae | Control | Algae |
| Corn (yellow) | 60.00 | 59.66 | 66.02 | 65.46 |
| Soybean meal (48.5% CP) | 29.50 | 22.00 | 22.00 | 14.60 |
| Meat meal | 5.00 | 5.00 | 5.00 | 5.00 |
| Corn gluten meal | 2.00 | 2.00 | 2.00 | 2.00 |
| Corn oil | 1.00 | 2.00 | 3.00 | 4.10 |
| Dicalcium phosphate | 0.50 | 0.20 | 0.20 | 0.06 |
| Limestone | 0.80 | 0.50 | 0.70 | 0.30 |
| Salt | 0.50 | 0.00 | 0.50 | 0.00 |
| Vitamin mix¹ | 0.25 | 0.25 | 0.25 | 0.25 |
| Mineral mix² | 0.15 | 0.15 | 0.15 | 0.15 |
| Defatted diatom | 0.00 | 7.50 | 0.00 | 7.50 |
| DL-Methionine | 0.22 | 0.31 | 0.08 | 0.14 |
| L-Lysine hydrochloride | 0.08 | 0.25 | 0.10 | 0.27 |
| L-Isoleucine | 0.00 | 0.09 | 0.00 | 0.09 |
| L-Threonine | 0.00 | 0.06 | 0.00 | 0.06 |
| L-Tryptophan | 0.00 | 0.03 | 0.00 | 0.02 |
| L-Arginine (free base) | 0.00 | 0.00 | 0.00 | 0.00 |
| Total ingredients | 100.00 | 100.00 | 100.00 | 100.00 |

Nutritional composition

| | | | | |
|---|---|---|---|---|
| ME (Kcal/g) | 3.00 | 3.01 | 3.21 | 3.21 |
| Protein, % | 23.1 | 21.3 | 20.4 | 18.2 |
| Fat, % | 4.1 | 5.3 | 6.3 | 7.5 |
| Fiber, % | 2.6 | 3.4 | 2.5 | 3.3 |
| Ca, % | 1.02 | 1.03 | 0.90 | 0.90 |
| P, % | 0.71 | 0.66 | 0.62 | 0.61 |
| P, % (available) | 0.46 | 0.45 | 0.40 | 0.41 |

¹Provided (in mg//kg of diet): CuSO₄•5H₂O, 31.42; KI, 0.046; FeSO₄•7H₂O, 224.0; MnSO₄•H₂O, 61.54; Na₂SeO₃, 0.13; ZnO, 43.56; Na₂MoO₄•2H₂O, 1.26.
²Provided (in IU/kg of diet): vitamin A, 6500; vitamin D₃, 3500; vitamin E, 25 and (in mg/kg of diet): riboflavin, 25; d-calcium pantothenate, 25; nicotinic acid, 150; cyanocobalamin, 0.011; choline chloride, 1250; biotin, 1.0; folic acid, 2.5; thiamine hydrochloride, 7.0; pyridoxine hydrochloride, 25.0; menadione sodium bisulfite, 5.0; and ethoxyquin, 66.

Experiment 1

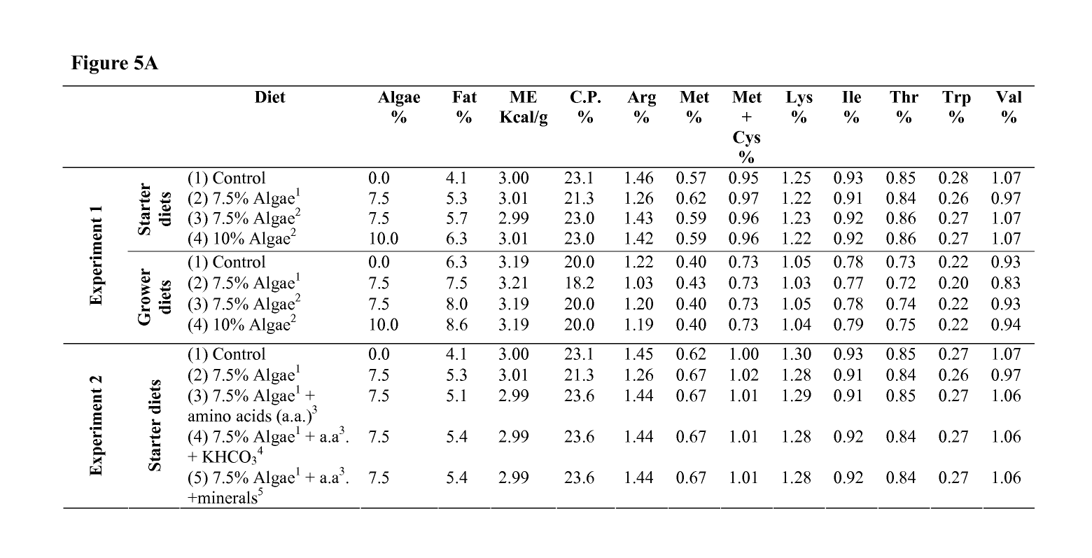

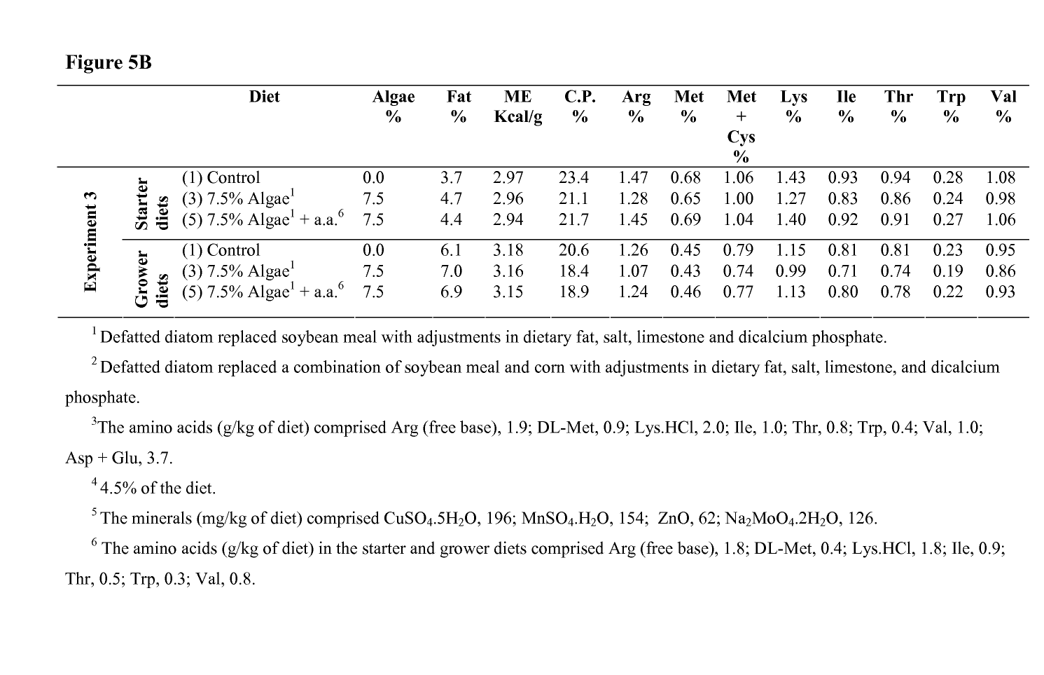

The objective was to determine whether DFA could replace a portion of SBM and/or corn in diets for broiler chicks. A total of 80 two-day-old chicks were used. Duplicate cages of 5 chicks per gender were assigned to 4 diets in a 2×4 factorial arrangement of treatments. The 4 dietary treatments comprised chick starter and grower controls (Diet 1) which were based on practical feed ingredients (Table 18), starter and grower diets containing 7.5% DFA substituting in part for SBM (Diet 2, Table 18), and starter and grower diets containing 7.5% (Diet 3) and 10% (Diet 4) DFA substituting in part for SBM and corn; a 1:3 and 1:4 SBM to corn mixture was replaced with DFA for Diet 3 and 4, respectively. All diets were formulated to be isoenergentic and to meet the requirements for all essential amino acids. The main differences in microalgal biomass inclusion and the calculated metabolizable energy, crude protein, and amino acid concentrations among diets of the three experiments are presented in FIGS. 5A-B. The calculations are based on chemical analyses of the DFA (Table 17) and published tables of the nutrient contents of conventional feedstuffs (National Research Council, Nutrient Requirements of Poultry, 9th revised Edition, National Academy Press, Washington D.C. (1994), which is hereby incorporated by reference in its entirety).

Experiment 2

This experiment was designed to determine if certain modifications of the diet containing 7.5% DFA replacing SBM would prevent the early growth depressions and lower body weight gain to feed intake ratio (“G:F”) that were observed in Experiment 1. A total of 100 two-day old chicks were used. Duplicate cages per gender of 5 chicks were assigned to 5 diets. Chicks received the control diet (Diet 1) or 7.5% DFA diet (Diet 2) with DL-methionine and L-lysine supplemented at 0.05% higher levels than in Experiment 1 (FIGS. 5A-B). Diet 3 was Diet 2 supplemented with arginine (Arg) and valine (Val) and the crude protein level was maintained similar to the control diet by supplementation with aspartic acid and glutamic acid in a 1.6:1 ratio. Diet 4 was Diet 3 with the addition of potassium bicarbonate to adjust the dietary electrolyte balance (Na+K−Cl) from 173 meq/kg in Diet 3 to 218 meq/kg in Diet 4. Diet 5 was formulated to contain added copper (Cu), manganese (Mn), molybdenum (Mo), and zinc (Zn), but otherwise was similar to diet 4. The duration of the experiment was 3 weeks.

Experiment 3

This 6 week experiment involving 180 three-day old male chicks was performed to confirm results of the previous experiments using 7.5% DFA replacing SBM and to determine whether the addition of proteases or amino acids improved broiler growth and G:F. Five treatments of 6 replicates of 6 chicks were used. The control diet (Diet 1) was similar to that of Experiment 1 except that it contained added threonine. Diet 2 was the control diet plus the inclusion of 0.06% commercial protease (Ronozyme ProAct, DSM Nutritional Products, Inc., Parsippany, N.J.). Diet 3 contained 7.5% DFA and was similar to Diet 2 of Experiment 1, and Diet 4 contained 7.5% DFA and 0.06% commercial protease. Diet 5 contained 7.5% DFA supplemented with Arg, Ile, Trp, and Val.

Blood Collection, Tissue Examination, and Biochemical Assays

After a 6 hour fast, blood was drawn from the wing veins of 2 birds per pen at week 6 in Experiment 1 and at 3 and 6 weeks in Experiment 3. Blood was held in ice during collection, centrifuged at 3000 g for 15 min, and stored at −20° C. until analyses. After blood sampling, birds were euthanized by cervical dislocation. In Experiment 1, the proventriculus (true stomach), ventriculus (gizzard), small intestine, and large intestine and cecum from each of 2 randomly selected chicks per cage were opened and examined for evidence of gross pathology. The koilin layer of the ventriculus was examined and removed for observation of the underlying tissue. To assess liver health and function, plasma alanine transaminase (“ALT”) activities were determined spectrophotometrically with the Infinity Alt liquid stable reagent (Thermo Electron Corporation) and plasma alkaline phosphatase (AKP) activities were analyzed by the method of Bowers & McComb (Bowers et al., “A Continuous Spectrophotometric Method for Measuring the Activity of Serum Alkaline Phosphatase,” Clin. Chem. 12:70-89 (1966), which is hereby incorporated by reference in its entirety). Plasma glucose level was determined spectrophotometrically with glucose assay kit GAG020 (Sigma-Aldrich, Sigma Chemical Co., St. Louis, Mo.). Plasma uric acid was analyzed with Infinity Uric Acid Liquid Stable Reagent (Thermo Scientific Corporation). Plasma and liver non-esterified fatty acid (NEFA), triglyceride (TG), and cholesterol (“CHOL”), indicators of lipid metabolism, were analyzed using commercial enzymatic kits (Wako Pure Chemicals Industries, Ltd., Richmond, Va.). All samples were analyzed in duplicates.

Statistical Analyses

Data were analyzed by one-way or two-way ANOVA with or without time-repeated measurements for determining significance of main effect using SPSS17.0. Mean comparisons were conducted with Duncan's method. The significance level for differences was P<0.05.

Results of Example 3

Experiment 1

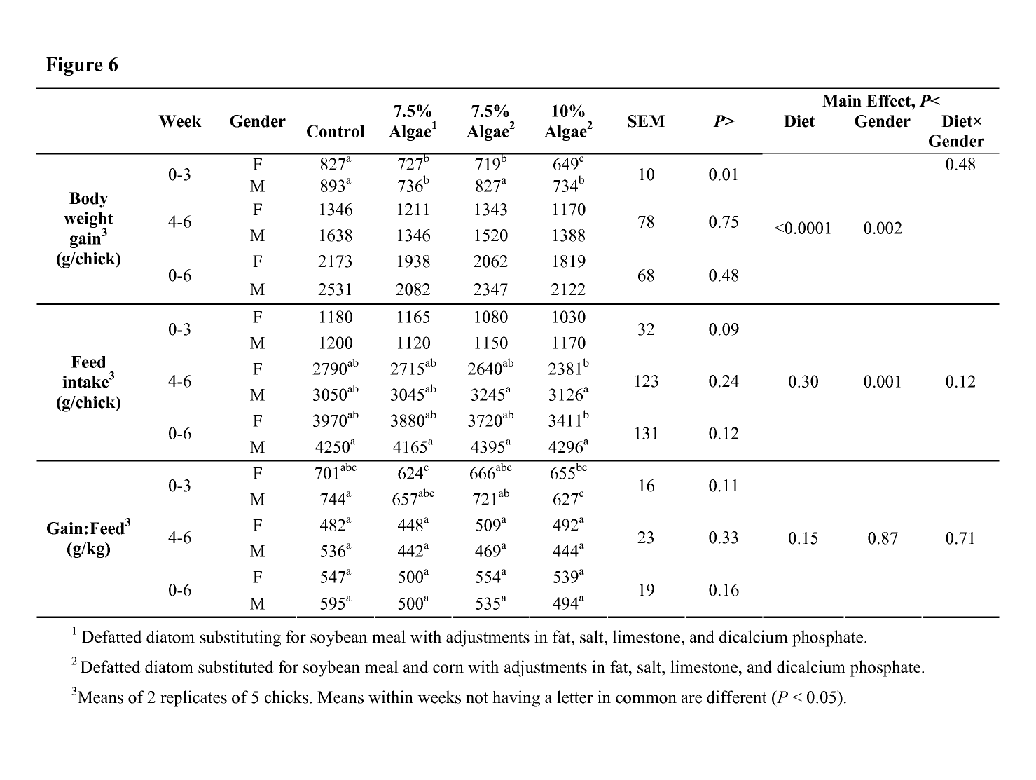

Body weight gain of chicks was affected by diet (P<0.0001) and gender (P<0.002) (FIG. 6). During the 0-3 week interval, all groups fed the DFA-containing diets except for those of males fed Diet 3 had lower (P<0.01) body weight gains than the control group. However, this adverse effect of DFA on body weight gain became statistically non-significant during the 4-6 week interval or for the cumulative 0-6 week period. Meanwhile, chicks fed the DFA-containing diets appeared to have lower feed intake (P=0.09) and gain:feed (P=0.11) than the control group during the 0-3 week interval. Gender affected feed intake (P<0.05), but not gain:feed. There was no anomaly or gross pathology in the proventriculus, ventriculus, and the intestinal tract.

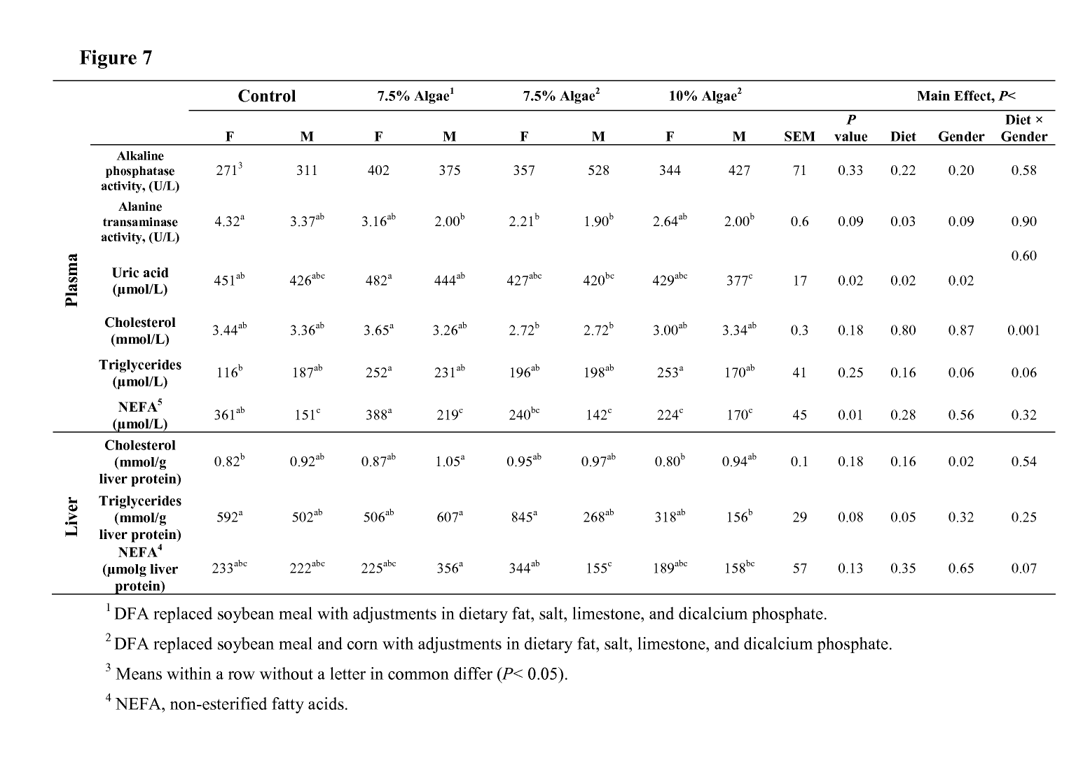

Diet exerted an overall effect (P<0.05) on plasma ALT activity and uric acid concentration (FIG. 7). Chicks fed DFA-containing diets tended to have lower activity of ALT than in chicks fed Diet 1, and the difference was significant (P<0.05) between the female chicks fed Diets 3 and 1. Plasma uric acid concentration was lower (P<0.05) in male chicks fed Diet 4 than Diet 2. None of the plasma uric acid values of males or females chicks fed the DFA-containing diets differed from those fed the control diet. There was an overall effect of gender (P<0.02) on plasma uric acid concentration, but no significant treatment mean difference was noticed. There was on overall effect of gender (P<0.02) on liver CHOL concentrations, and also an overall effect of diet (P<0.05) on plasma TG concentrations along with a significant difference between male chicks fed Diets 4 and 2.

Experiment 2

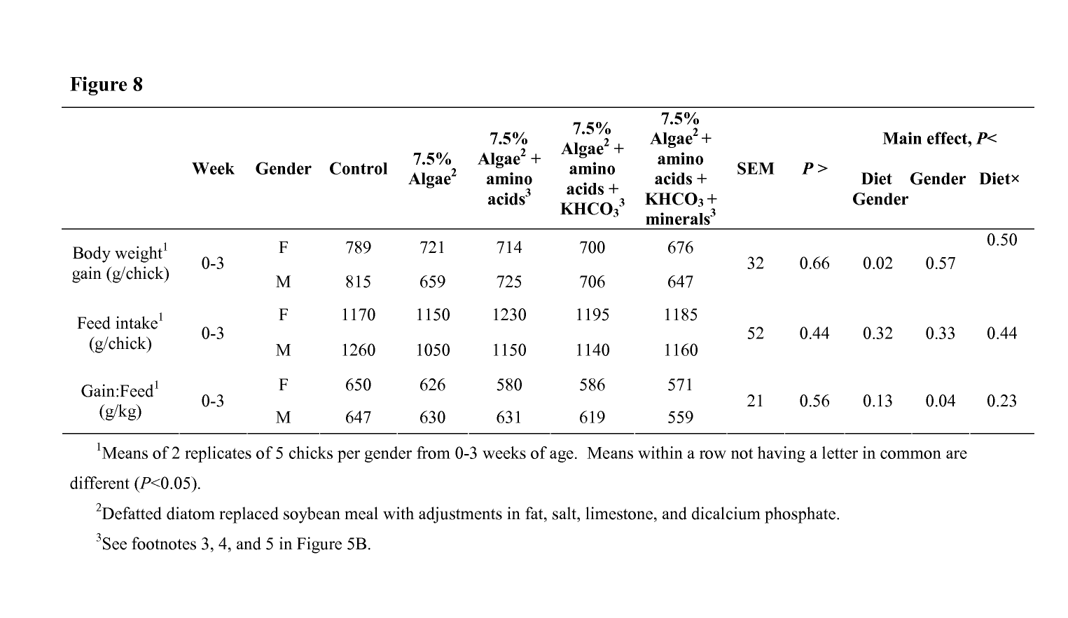

There was an overall effect of diet (P<0.02) on body weight gain (FIG. 8), although the decreases by any given DFA-containing diet over the control diet reached no statistically significant level. Gain:feed was affected by gender (P<0.04) and showed a decline trend (P=0.13) in response to the DFA-containing diets compared with the control diet. Supplementing the DFA-containing diet (Diet 2) with amino acids, KHCO₃, and minerals (Diets 3-5) produced no additional benefit to body weight gain, feed intake, or gain:feed. Three male chicks fed Diet 2 in Experiment 2 exhibited swollen hocks and difficulty walking at 3 weeks of age.

Experiment 3

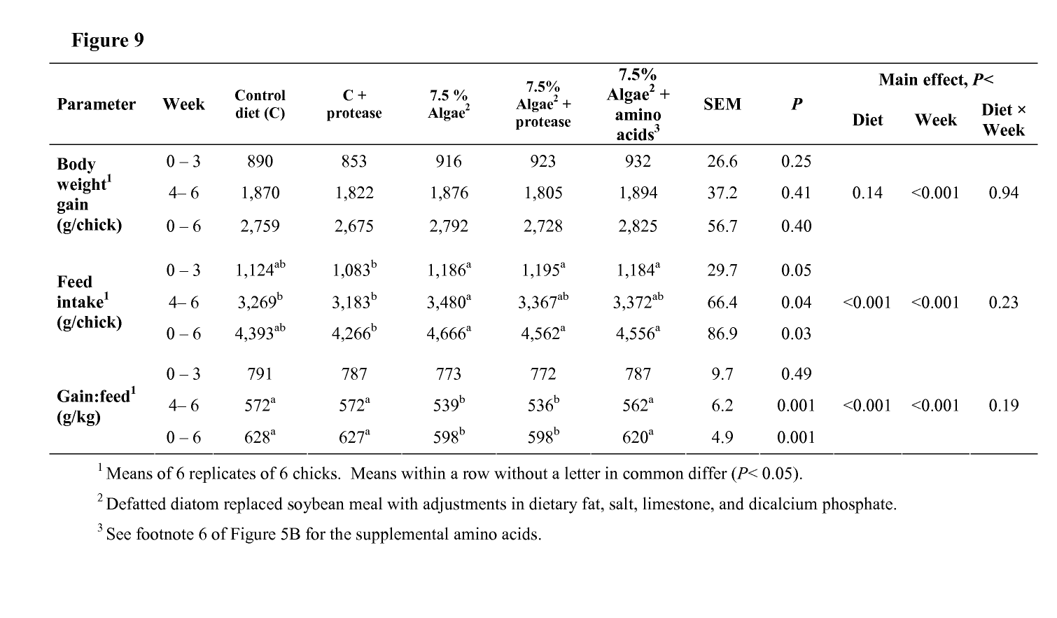

Diet affected (P<0.001) feed intake and gain:feed, but not body weight gain (FIG. 9). The feed intakes of chicks fed the three DFA-containing diets were greater (P<0.05) than those fed Diet 2 during the 0-3 week interval and the 0-6 week period. There was no difference among dietary groups in gain:feed during the 0-3 week interval, the ratios during the 4-6 week and 0-6 week periods were lower (P<0.05) in chicks fed 7.5% DFA diet (Diet 3) and the 7.5% DFA plus protease (Diet 4) than in those fed the diets (1 and 2) without DFA and the DFA-containing diet supplemented with amino acids (Diet 5). Chicks fed Diet 5 had similar gain:feed ratios to those fed Diets 1 and 2.

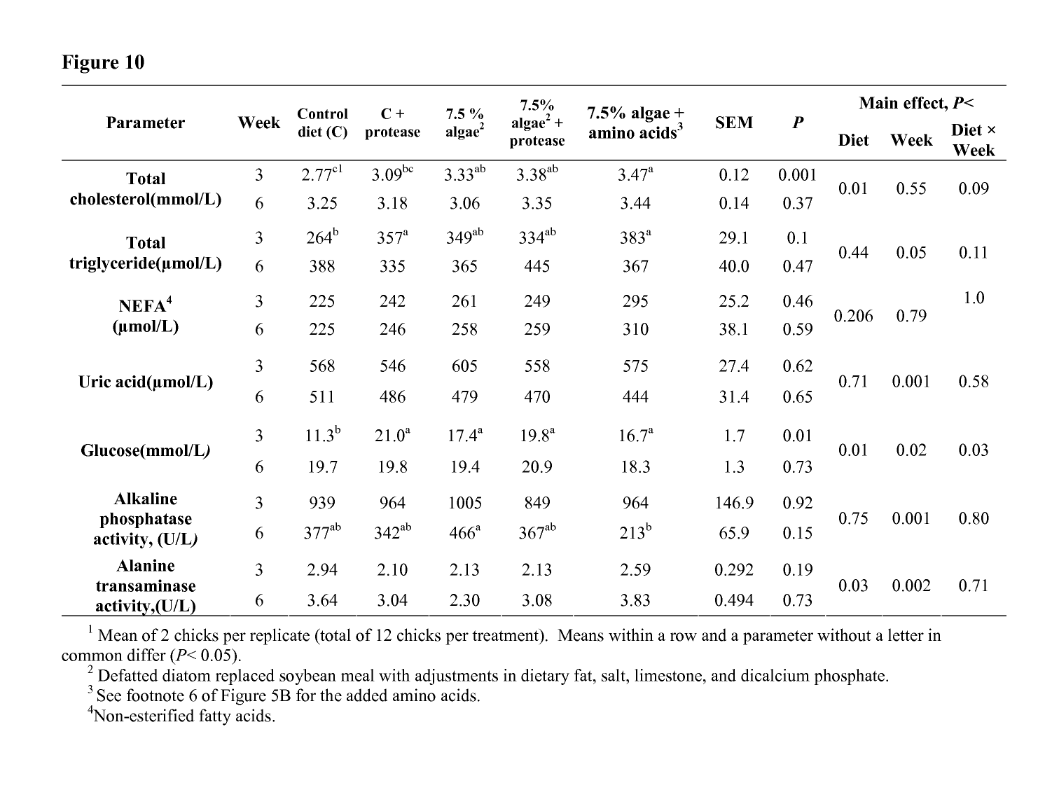

Three of the biochemical measures of plasma:CHOL, glucose, and ALT, were affected by diet (P<0.05) (FIG. 10). There were effects (P<0.05) of time (3 or 6 weeks) on plasma total triglyceride, uric acid, glucose, AKP, and ALT (P<0.05). Total plasma CHOL was higher at 3 weeks in the chicks fed diets containing DFA than in chicks fed the control diet (P<0.05). Plasma glucose was higher (P<0.05) at 3 weeks in chicks fed the DFA-containing diets than in those fed the control diet. Despite an overall effect of diet (P<0.03) on plasma ALT activity, there was no significant difference between any treatment means. Plasma uric acid concentration and AKP activity of chicks were lower (P<0.05) at 6 weeks than at 3 weeks.

Discussion for Example 3

The overall finding of Experiment 1 indicated that inclusion of the defatted diatom microalgal Staurosira sp. biomass from biofuel production, at 7.5% of diet in replacing the same amount of SBM and corn had an adverse effect on growth in the 0-3 week interval, but did not significantly affect body weights by the end of the experiment. In contrast, the 10% of inclusion or the 7.5% of inclusion in replacing SBM alone decreased body weight gain, feed intake, and gain:feed, especially during the 0-3 week period. The DFA used in the present study contained 19% crude protein (“CP”) on an “as fed” basis, and had no major limitations of amino acids as a source of feed protein. However, it was expected that its substitution for SBM would require a supplementation of the chick diets with Met and Lys. Since the CP content of the DFA was lower than the CP (47.5%) of SBM, but was greater than the CP (8.5%) of corn (National Research Council, Nutrient Requirements of Poultry, 9th revised Edition, National Academy Press, Washington D.C. (1994), which is hereby incorporated by reference in its entirety), substitution of DFA for a mixture of SBM and corn had little effect on the dietary CP level. However, the supplementation of the DFA for SBM resulted in a lower level of CP in the diet compared to the control diet. Thus, this depressed growth performance of chicks might be associated with the lower protein level, an insufficient dietary level of one or more indispensable amino acids, lower protein digestibility, or a combination of these factors.

The notion on amino acid limitation in the DFA-containing diets was supported in part by the results of Experiment 3 in which the decreased gain:feed due to the 7.5% DFA inclusion was prevented by the addition of Met, Lys, Ile, Thr, Trp, and Val to the diet. Apparently, one or more of these amino acids must have been limiting in the DFA-containing diets, contributing to the reduction in feed use efficiency compared with the control diet. Meanwhile, the dietary levels of Met and Lys were raised by 0.05 percentage points in Experiment 2 as compared to Experiment 1 to ensure that the levels would be adequate for rapidly growing broilers. Sulfur-containing amino acids (Met or Cys) and Lys are generally the first and second most limiting amino acids in practical diets based on corn and soybean for broilers (Emmert et al., “Use of the Ideal Protein Concept for Precision Formulation of Amino Acid Levels in Broiler Diets,” J Appl. Poult. Res. 91:683-692 (1997), which is hereby incorporated by reference in its entirety). The inability to ascertain the specific effects of amino acid supplementation in Experiment 2 might have been due to the increased dietary levels of Met and Lys. The increase in supplementation could have been sufficient to mitigate a mild deficiency of one or both amino acids. The further addition of Arg and Val to raise the levels of these indispensable amino acids and the addition of glutamic acid and aspartic acid to raise the CP level to 23.5% did not enhance growth rate or feed efficiency. These results suggest that Arg, Val, and CP were not limiting. However, the addition of protease to the control diet or the DFA-containing diet in Experiment 3 did not improve broiler growth rate or gain:feed. The failure of the protease to improve performance, while amino acid supplements were effective, may indicate that proteins in the DFA were not hydrolyzed by the protease and/or did not require additional proteolysis. Algaenan, a class of proteins in some algae, is reported to be resistant to enzymatic hydrolysis (Nguyen et al., “Preservation of Algaenan and Proteinaceous Material During the Oxic Decay of Botryococcus braunii as Revealed by Pyrolysis-Gas Chromatography/Mass Spectroscopy and 13C NMR Spectroscopy,” Organic Geochem. 34:483-497 (2003), which is hereby incorporated by reference in its entirety). Proteins of this class have been detected in many, but not all, algae (Kodner et al., “Phylogenetic Investigation of the Aliphatic, Non-Hydrolyzable, Biopolymer Algaenan, with a Focus on the Green Algae,” Organic Geochem. 40(8):854-862 (2009), which is hereby incorporated by reference in its entirety). It is unknown whether they are present in the DFA used in the present study.

The dietary balance of the monovalent minerals sodium (Na), potassium (K), and chlorine (Cl) influences the growth and skeletal development of broiler chickens (Lipstein et al., “The Nutritional Value of Sewage-Grown Samples of Chlorella and Micractinium in Broiler Diets,” Poult. Sci. 62:1254-1260 (1983); Sauveur et al., “Interrelationship Between Dietary Concentrations of Sodium, Potassium and Chloride in Laying Hens,” Br. Poult. Sci. 19:475-485 (1978), which are hereby incorporated by reference in their entirety). The optimum dietary electrolyte balance (Na+K−Cl) for broilers is near 200 meq/kg of feed for maximum growth and minimum incidence and severity of tibial dyschondroplasia (Mongin et al., “In: Growth and Poultry Meat Production,” Boorman, K. N.; Wilson, B. J. (Ed), Interrelationships Between Mineral Nutrition, Acid-Base Balance, Growth and Cartilage Abnormalities, British Poultry Science, Ltd, Edinburgh, pgs. 235-247 (1977), which is hereby incorporated by reference in its entirety). Raising the electrolyte balance from 173 to 218 meq/kg in the DFA-containing diet did not improve broiler growth or feed utilization. Therefore, electrolyte balance probably was not a limiting factor for the adverse effect of the DFA-containing diet. Because diatoms contain amorphous Si, and this element interacts in biological systems with divalent minerals such as iron (Fe), molybdenum (Mo), copper (Cu), and zinc (Zn), (Emerick et al., “Interactive Effects of Dietary Silicon, Copper, and Zinc in the Rat,” J. Nutr. Biochem. 1:35-40 (1990); National Research Council, Mineral Tolerance of Animals, Second revised Edition, Chapter 26, Silicon, National Academy Press, Washington, D.C. pgs. 348-354 (2005), which are hereby incorporated by reference in their entirety) the dietary levels of trace minerals were increased in Diet 5. Likewise, these elevations of trace minerals were ineffective in restoring growth or feed utilization.

Although some minor differences in plasma uric acid, glucose, and lipid concentrations were observed, the plasma biomarkers did not reveal any indication of adverse effects of the DFA on metabolism. Plasma AKP and ALT, indicators of liver health and function, were not increased by the inclusion of the DFA in the diet. In contrast, the 10% level of DFA tended to decrease the uric acid concentration in the males at 6 weeks in Experiment 1. The males tended to grow faster and ingested more feed than the females. These results were consistent with known gender differences in growth rate, efficiency of feed utilization for body weight gain, and nitrogen retention (Hernandez et al., “Effect of Low Protein Diets and Single Sex on Production Performance, Plasma Metabolites, Digestibility, and Nitrogen Excretion in 1- to 48-day-old Broilers,” Poult. Sci. 91:683-692 (2012), which is hereby incorporated by reference in its entirety). To determine if algal inclusion affected nutrient metabolism, measures of plasma and liver lipid and carbohydrate metabolism were examined. Plasma glucose concentrations were elevated at 3 weeks in chicks fed the DFA-containing diets in Experiment 3, perhaps indicating a higher availability of carbohydrates in the DFA than in the replaced SBM. Chicks fed the DFA-containing diets tended to have higher plasma total CHOL and TG concentrations at 3 weeks than chicks fed the control diet. This increase might also reflect increased carbohydrate availability from DFA and the fact that the DFA-containing diets had higher crude fat contents than the control diets. The metabolizable energy value that was used in the dietary formulations in these experiments was estimated from the fat and protein content of the DFA. It is possible that it was an underestimate, resulting in higher dietary energy concentration in the DFA-containing diets than what was targeted.

Two observations may lead to further research on the use of the DFA in broiler feeds. First, the chicks fed the DFA-containing diets had, by visual observation, an increased volume and wetness of excreta as compared to the volume and moisture of excreta from chicks fed the control diet. The ash fraction of the DFA accounted for nearly 45% of the weight of the biomass “as fed” and contained substantial amounts of Na, K, Mg, Fe, Cl. The increased wet droppings undoubtedly were a consequence of the ash content of the defatted diatom. Excessive output of excreta is a potential liability of the diatom feeding. Further processing to reduce the ash content of the DFA would improve this co-product as a feed ingredient for poultry and other animals. The second observation of potential concern was the incidence of hock disorder in males fed the basic 7.5% DFA diet in Experiment 2. The disorder was not observed in Experiments 1 and 3. Given the propensity of broiler chicks to develop leg abnormalities (Waldenstedt, L., “Nutritional Factors of Importance for Optimal Leg Health in Broilers: A Review,” Anim. Feed Sci. Technol. 126(3-4):291-307 (2006), which is hereby incorporated by reference in its entirety), however, an investigation of skeletal development in chicks fed diets containing the DFA is advisable.

In summary, the results of the present study indicate that the DFA of Staurosira sp. could be used as a protein and energy source in broiler diets. The inclusion level of 7.5% for replacing a mixture of corn and SBM was well tolerated by broilers. The same inclusion level for replacing SBM alone was also feasible when certain amino acids were supplemented. Strikingly, the latter inclusion of 7.5% DFA in broiler diets to replace SBM will spare over 2.4 million metric tons of soybean for human consumption annually.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An animal feed composition comprising:
one or more grains in an amount totaling 50-70% w/w of the composition;
a non-algal protein source in an amount totaling 15-30% w/w of the composition;
algae in an amount totaling 3-15% w/w of the composition;
an oil heterologous to the algae in an amount totaling 0.5-15% w/w of the composition;
an inorganic phosphate source in an amount totaling up to 1.5% w/w of the composition;
a sodium source in an amount totaling up to 0.5% w/w of the composition; and
one or more amino acids selected from the group consisting of lysine, threonine, isoleucine, tryptophan, and methionine in an amount totaling up to 0.5% w/w of the composition.

2. The composition of claim 1, wherein the one or more grains comprises maize, wheat, rice, sorghum, oats, potato, sweet potato, cassava, DDGS, and combinations thereof.

3. The composition of claim 1, wherein the non-algae protein source comprises soybean, fishmeal, cottonseed meal, rapeseed meal, meat meal, plasma protein, blood meal, and combinations thereof.

4. The composition of claim 1, wherein the algae comprises full-fat algae.

5. The composition of claim 4, wherein the oil heterologous to the algae is present in the composition in an amount totaling 0.5-5% w/w of the composition.

6. The composition of claim 1, wherein the algae comprises de-fatted algae.

7. The composition of claim 6, wherein the oil heterologous to the algae is present in the composition in an amount totaling 3-15% w/w of the composition.

8. The composition of claim 1, wherein the oil heterologous to the algae comprises corn oil.

9. The composition of claim 1, wherein the algae comprises diatom algae.

10. The composition of claim 1, wherein the phosphate source comprises dicalcium phosphate.

11. The composition of claim 1 further comprising one or more of the following:
plasma protein in an amount totaling 0.5-3.0% w/w of the composition;
an inorganic calcium source in an amount totaling 0.1-10% w/w of the composition;
a vitamin/mineral mix in an amount totaling 0.1-1% w/w of the composition, wherein the vitamin/mineral mix comprises one or more trace minerals;
an inorganic magnesium source in an amount totaling 0.01-0.1% w/w of the composition; and
an antibiotic in an amount totaling 0.01-0.1% w/w of the composition.

12. The composition of claim 11, wherein the one or more trace minerals are selected from Cu, Se, Zn, I, Mn, Fe, and Co.

13. An animal feed supplement comprising:
algae;
an inorganic phosphate source in an amount (w/w) of algae (1-25):inorganic phosphate (1-2);
a sodium source in an amount (w/w) of algae (1-25):sodium (0.1-0.6); and
one or more amino acids selected from the group consisting of lysine, threonine, isoleucine, tryptophan, and methionine in an amount (w/w) of algae (1-25):amino acids (3-5).

14. The animal feed supplement of claim 13, wherein the algae comprises full-fat algae.

15. The animal feed supplement of claim 13, wherein the algae comprises defatted algae.

16. The animal feed supplement of claim 13 further comprising:
oil heterologous to the algae in an amount (w/w) of algae (1-25):oil (3-15).

17. The animal feed supplement of claim 16, wherein the oil heterologous to the algae comprises corn oil.

18. The animal feed supplement of claim 13, wherein the algae comprises diatom algae.

19. The animal feed supplement of claim 13, wherein the inorganic phosphate source comprises dicalcium phosphate.

20. The animal feed supplement of claim 13 further comprising one or more of the following:
plasma protein in an amount (w/w) of algae (1-25):plasma protein (1-5);
an inorganic calcium source in an amount (w/w) of algae (1-25):calcium (1-4);
a vitamin/mineral mix comprising trace minerals, wherein the vitamin/mineral mix is provided in an amount (w/w) of algae (1-25):vitamin/mineral mix (0.1-2);
an inorganic magnesium source in an amount (w/w) of algae (1-25):magnesium (0.01-0.1); and
an antibiotic in an amount (w/w) of algae (1-25):antibiotic (0.01-0.1).

21. The animal feed supplement of claim 20, wherein the one or more trace minerals are selected from Cu, Se, Zn, I, Mn, Fe, and Co.

22. A method of feeding an animal, said method comprising:
administering to an animal the animal feed composition of claim 1.

23. The method of claim 22, wherein the animal is selected from the group consisting of a ruminant, poultry, swine, aquaculture, pet, dog, cat, horse, zoo animal, mouse, rat, rabbit, guinea pig, and hamster.

24. The method of claim 23, wherein the animal is selected from the group consisting of a laying hen, a broiler chicken, and a weanling pig.

25. A method of feeding an animal, said method comprising:
administering to an animal an animal feed in combination with the animal feed supplement of claim 13.

26. The method of claim 25, wherein the animal is selected from the group consisting of a ruminant, poultry, swine, aquaculture, pet, dog, cat, horse, zoo animal, mouse, rat, rabbit, guinea pig, and hamster.

27. The method of claim 26, wherein the animal is selected from the group consisting of a laying hen, a broiler chicken, and a weanling pig.

28. A method of improving the feed efficiency of an animal, said method comprising:
administering to an animal an animal feed in combination with the animal feed supplement of claim 13 under conditions effective to cause a 3-15% decrease in plasma uric acid levels in the animal relative to such animal receiving the animal feed without the animal feed supplement, thereby improving the feed efficiency in the animal.

29. The method of claim 28, wherein the animal is selected from the group consisting of a ruminant, poultry, swine, aquaculture, pet, dog, cat, horse, zoo animal, mouse, rat, rabbit, guinea pig, and hamster.

30. The method of claim 29, wherein the animal is selected from the group consisting of a laying hen, a broiler chicken, and a weanling pig.

31. A method of improving the feed efficiency of an animal, said method comprising:
administering to an animal the animal feed composition of claim 1 under conditions effective to cause a 3-15% decrease in plasma uric acid levels in the animal relative to such animal receiving an animal feed other than the animal feed composition, thereby improving the feed efficiency in the animal.

32. The method of claim 31, wherein the animal is selected from the group consisting of a ruminant, poultry, swine, aquaculture, pet, dog, cat, horse, zoo animal, mouse, rat, rabbit, guinea pig, and hamster.

33. The method of claim 32, wherein the animal is selected from the group consisting of a laying hen, a broiler chicken, and a weanling pig.

34. In an animal feed, the improvement comprising:
algae in an amount effective to decrease uric acid levels in plasma in an animal by 3-15% after consuming the animal feed, thereby improving feed efficiency in the animal.

35. The animal feed of claim 34, wherein the animal is selected from the group consisting of a ruminant, poultry, swine, aquaculture, pet, dog, cat, horse, zoo animal, mouse, rat, rabbit, guinea pig, and hamster.

36. The animal feed of claim 35, wherein the animal is selected from the group consisting of a laying hen, a broiler chicken, and a weanling pig.

37. The animal feed of claim 34, wherein the algae comprises full-fat algae.

38. The animal feed of claim 34, wherein the algae comprises de-fatted algae.
